(19) United States
(12) Patent Application Publication
(10) Pub. No.: US 2024/0245664 A1
(43) Pub. Date: Jul. 25, 2024

(54) METHODS OF USING USP15 INHIBITORS

(71) Applicant: The Curators of the University of Missouri, Columbia, MO (US)

(72) Inventor: Thang VAN NGUYEN, Columbia, MO (US)

(21) Appl. No.: 18/690,470

(22) PCT Filed: Sep. 7, 2022
(86) PCT No.: PCT/US2022/076022

§ 371 Date: Mar. 8, 2024

Related U.S. Application Data
(60) Provisional application No. 63/241,555, filed on Sep. 8, 2021.

(51) Int. Cl. A61K 31/44; A61K 31/4015; A61K 31/454; A61K 45/06; C12N 15/113; G01N 33/574

(52) U.S. Cl. CPC A61K 31/44; A61K 31/4015; A61K 31/454; A61K 45/06; C12N 15/1137; G01N 33/57407; C12N 2310/11; C12N 2310/14; C12N 2310/141; C12N 2310/531; G01N 2474/20

(57) ABSTRACT

The present disclosure is generally directed to compositions and methods related to USP15 inhibitors. More particularly, the present disclosure relates to methods of regulating CRL4^{CRBN}-USP15 pathway (previously referred to as the CRL4^{CRBN}-p97 pathway) and glutamine synthetase levels and methods of treating diseases such as cancer via administration of USP15 inhibitors.

### CROSS REFERENCE TO RELATED APPLICATIONS

This application is a U.S. National Phase application of PCT/US2022/076022 (published as WO 2023/039405), filed on Sep. 7, 2022, which claims the benefit of U.S. Provisional Application No. 63/241,555, filed Sep. 8, 2021, the contents of each of which are incorporated herein in their entireties.

### INCORPORATION OF SEQUENCE LISTING

A computer readable form of the Sequence Listing XML containing the file named “3513285.0365_Sequence_Listing.xml,” which is 2,666 bytes in size (as measured in MICROSOFT WINDOWS® EXPLORER) and are herein incorporated by reference. This Sequence Listing consists of SEQ ID NOs: 1-2.

### FIELD OF THE DISCLOSURE

The present disclosure provides compositions and methods related to USP15 inhibitors. More particularly, the present disclosure is directed to methods of regulating the CRL4^{CRBN}-USP15 pathway (previously referred to as the CRL4^{CRBN}-p97 pathway) and glutamine synthetase levels and methods of treating diseases such as cancer via administration of USP15 inhibitors.

### BACKGROUND OF THE DISCLOSURE

Cancer and the CRL4^{CRBN}-USP15 (Previously CRL4^{CRBN}-p97) Pathway

Cancer is the second leading cause of death in the United States. Despite recent advances in many cancer therapies, drug resistance to available medicines remains one of the biggest challenges in the effort to cure cancer. For example, multiple myeloma (MM) accounts for 1.8% of all new cancer cases in the United States with a 5-year survival rate of only 55.6%. Ubiquitylation of protein substrates is catalyzed by the ubiquitin-specific E1, E2, and E3 ligases and can be reversed by deubiquitinating enzymes (DUBs). The key components of the ubiquitin-proteasome system (UPS), including over 600 E3 ligases, ˜100 DUBs and the proteasome has recently emerged as therapeutic targets for treatment of cancer and other diseases. However, there are fundamental gaps in the knowledge and understanding of how E3s and DUBs control degradation of individual protein substrates.

Thalidomide was prescribed to pregnant women as a sedative to treat morning sickness in the late 1950s. It was withdrawn from the market in the early 1960s, when it was found to be a cause of severe birth defects. Recently, thalidomide and its immunomodulatory derivatives (IMiDs) lenalidomide and pomalidomide, have been used for the treatment of multiple myeloma (MM) and other hematologic malignancies. Other thalidomide analogs, now known as the Cereblon E3 ligase modulators (CELMoDs), including CC-122, CC-220 and CC-885, have been developed to target pathogenic proteins for degradation via a molecular glue mechanism. For example, CC-885 induces degradation of the translation termination factor GSPT1, a CRL4^{CRBN} neo-substrate. In addition, CRBN is also a target for the development of proteolysis-targeting chimaera (PROTAC) technology, which relies on linking a drug that binds to a protein of interest to IMiDs. Many CRBN-based PROTACs, including the potent BRD4 protein degrader dBET1, have been developed for the degradation of target proteins in cancer and other human diseases. Despite recent advances in the field, many questions apart from clinical efficacy of IMiDs remain unknown. For example, CRBN is required for the action of IMiDs, but CRBN protein levels do not correlate with intrinsic sensitivity or resistance to IMiDs in MM cell lines, suggesting that other factors play a critical role in regulating the mechanisms underlying sensitivity and/or resistance to IMiDs.

However, the exact mechanisms of action of thalidomide that bring about its harmful and beneficial effects are not well understood. Recently, it was reported that cereblon (CRBN) protein is a primary target of thalidomide teratogenicity and that CRBN is key target of antitumor activities, and the CRL4^{CRBN}-p97 pathway was only recently discovered.

Although the immunomodulatory drugs (IMiDs) thalidomide, lenalidomide, and pomalidomide have revolutionized the treatment of patients with multiple myeloma (MM) and other hematologic malignancies, almost all patients eventually develop resistance to IMiDs. Cereblon (CRBN) protein is a direct target for thalidomide teratogenicity and antitumor activity of IMiDs. The binding of IMiDs to CRBN promotes the recruitment of neo-substrates including Ikaros (IKZF1) and Aiolos (IKZF3) and casein kinase 1a (CK1a), leading to their ubiquitylation and subsequent degradation. CRBN is required for antitumor activity of IMiDs, but its expression levels do not correlate with intrinsic sensitivity or resistance to IMiDs in MM cells, suggesting that another factor is essential for regulating the mechanisms of IMiD sensitivity and resistance. It was discovered the CRL4^{CRBN}-p97 pathway was required for degradation of GS and neo-substrates (such as GS, IKZF1, IKZF3, CK1α, RNF166, GSPT1 and BRD4). The crystal structure of CRBN bound to DDB1 and IMiDs revealed that the glutarimide rings of IMiDs bind a hydrophobic pocket in the thalidomide-binding domain of CRBN, whereas the phthalimide portion is largely exposed to solvent, critical for the recruitment of neo-substrates to the complex. Multiple neo-substrates such as ZNF692, SALL4 and RNF166 have been reported to be targeted for IMiD-induced ubiquitylation and subsequent degradation by the proteasome. Despite these developments, molecular control of the CRL4^{CRBN}-p97 pathway to manage degradation of target proteins is poorly understood.

CRBN-based platforms have been used to develop small molecules to target pathogenic proteins for degradation via molecular glue and PROTAC mechanisms. However, the most fundamental question in the field is how the CRL4^{CRBN}-p97 pathway manages to degrade target proteins.

Deubiquitylating enzymes (DUBs) play major roles in diverse cellular processes and altered DUB activity is associated with human diseases including cancer. There are approximately 100 DUBs encoded in the human genome, divided into five families based on their specific structural domains: ubiquitin C-terminal hydrolases (UCHs), ubiquitin-specific proteases (USPs), ovarian tumor proteases (OTUs), Josephins, and JAB1/MPN/MOV34 metalloenzymes (JAMMs). Genetic alterations and overexpression of USP15 have been reported in many human cancers in particular GBM, breast and ovarian cancer, leukemia and lymphomas, and MM. High expression of USP15 was significantly correlated with poor survival rate within the pan-cancer patient cohort, representing a key feature of oncogene activity. At the molecular level, USP15 has been shown to deubiquitylate and stabilize protein substrates in different signaling pathways such as TGF-β, MDM2 and NF-κB. Since Usp15^{-/-} mice were viable, targeting USP15 in cancer could achieve major advantages for an optimal therapeutic window. However, the molecular mechanism underlying the oncogenic activity of USP15 remains elusive.

### Glutamine Synthetase (GS)

Metabolic reprogramming is now recognized as a hallmark of cancer. Glutamine plays important roles in many cellular processes, including oxidative metabolism and ATP generation, biosynthesis of proteins, lipids and nucleic acids, as well as regulation of mTOR signaling pathway and autophagy. Glutamine synthetase (GS) is the only enzyme that is capable of the de novo synthesis of glutamine and detoxifies glutamate and ammonia. Mutations and deregulation of GS have been linked to human diseases, including congenital glutamine deficiency, Alzheimer's disease, and cancers in particular glioblastoma (GBM).

Recent studies highlighted the importance of glutamine metabolism in metabolic reprogramming. Activation of oncogenes such as MYC amplification and/or loss of tumor suppressor genes including p53 directly mediate the reprogramming of glutamine metabolism by selectively activating their downstream signaling or metabolic pathways. As a result, tumor cells display “glutamine addiction” and require excessive amounts of exogenous glutamine to generate building blocks and energy for their growth and survival. In striking contrast, some tumor cell lines express high levels of GS enzyme and can synthesize sufficient glutamine to fulfill glutamine requirements for nucleotide and protein synthesis.

Prior studies also demonstrated that bacterial GS is regulated by reversible adenylylation. In contrast to the well-defined regulation of bacterial GS, the molecular mechanism underlying the regulation of GS activity in mammals is poorly understood. Almost sixty years ago, it was reported that the mammalian GS is inactivated by extracellular glutamine. Subsequent work done prior to the discovery of ubiquitin-proteasome system (UPS), suggested that glutamine stimulates GS degradation through an unknown mechanism. Recently, it was reported that GS and AMPKγ are endogenous substrates of CRL4^{CRBN}. Notably, p97 acts downstream of CRL4^{CRBN} to promote disassembly of ubiquitylated GS subunits, which are subsequently degraded by the proteasome. It was also recently found that endogenous GS protein levels are negatively regulated by glutamine through a feedback loop involving CRL4^{CRBN20}. However, it remains unknown how cells sense extracellular glutamine levels to control GS stability in the CRL4^{CRBN}-p97 pathway.

The molecular events that take place at each step of the pathway are not well understood. More recently, it was shown that valosin-containing protein (VCP)/p97, is required for GS degradation. p97 extracts ubiquitylated GS subunits from the decamer so that they can be degraded by the proteasome. Many important questions about this process remain unanswered. Two questions are how cells sense glutamine levels to control GS acetylation, and whether acetylation may be a general mechanism for targeting substrates to CRBN.

Both lysine 11 and lysine 14 on GS (KxxK motif) are acetylated, resulting in CRBN binding, CRBN-dependent ubiquitylation and subsequent degradation by the proteasome. However, the current understanding of acetylation-mediated recognition between substrate and CRBN is limited, since GS is the only endogenous substrate for CRBN has been well characterized. Because CRBN is a key target of IMiDs used to treat cancer, understanding how CRBN recognizes acetylated substrates and how this is influenced by IMiDs could create new opportunities for IMiD therapy.

In the Crbn-knockout mouse model, Crbn deficiency protects mice from obesity, fatty liver, and insulin resistance caused by high fat intake, suggesting that CRBN may regulate multiple substrates involved in body metabolism and energy homeostasis. Moreover, previous acetylome studies indicated that reversible lysine acetylation plays an important role in regulating metabolic enzymes.

Histone acetylation plays important roles in regulating global chromatin architecture and gene transcription. Previous studies indicated that actyl-CoA, a downstream metabolite of carbon sources, contributes to increased histone acetylation at genes important for cellular growth in yeast and human cells. However, the link between glutamine metabolism and epigenetic regulation of gene transcription in cancer remains unknown. It was recently observed that glutamine activates p300 to acetylate GS at lysines 11 and 14, raising the possibility that glutamine and its metabolites may induce global protein acetylation, especially histone acetylation, thereby activating transcription of genes involved in cellular growth and proliferation.

Overexpression of GS has been implicated in the development of different types of cancer, including Hepatocellular carcinoma (HCC), breast and glioblastoma. In addition, bioinformatic analysis of cell lines from The Cancer Cell Line Encyclopedia shows that many other types of cancer such as leukemia, chronic myelogenous leukemia (CML), acute myelogenous leukemia (AML) and prostate express higher GS mRNA levels. Interestingly, high GS expression may facilitate tumorigenesis, in that transgenic zebrafish, expressing an activated form of the Hippo pathway effector Yap1 (YAP) that reprograms glutamine metabolism through activation of GS expression, develop enlarged livers and are prone to liver tumorigenesis. However, the Hippo pathway can activate many other downstream target genes that may contribute to tumor development. Thus, the in vivo function of GS in tumorigenesis remains to be determined.

Compelling evidence suggests that cancer cells implement metabolic reprogramming by regulating transcription and post-translational modifications (PTMs) of metabolic enzymes. A recent study analyzing mRNA profiles of 1,454 metabolic enzymes across 1,981 tumors spanning 19 cancer types to identify enzymes that are consistently differentially expressed revealed that many enzymes and pathways are consistently overexpressed or downregulated across a large number of different cancer types. Interestingly, the authors also found that among the top 50 consistently overexpressed enzymes identified, 26 are essential for tumorigenesis in vitro and in vivo in breast cancer cells. However, this study only addressed changes in enzyme expression at the mRNA level, and did not seek to identify metabolic enzymes that may be dysregulated in cancer by alterations in PTMs such as ubiquitylation, acetylation, SUMOylation and phosphorylation. It has recently been demonstrated that PTMs play an essential role in regulating metabolic enzymes by multiple mechanisms, including via control of enzymatic activity, and by influencing protein stability.

Thus, it remains unknown how metabolism controls GS stability in the CRL4^{CRBN}-p97 pathway and how modulating metabolism and these pathways could impact cancer treatment.

### SUMMARY OF THE DISCLOSURE

One aspect of the disclosure is directed to a method of treating cancer in a subject in need thereof, the method comprising administering to the subject in need thereof a therapeutically effective amount of a USP15 inhibitor and at least one chemotherapeutic drug.

Another aspect of the disclosure is directed to methods of diagnosing and treating resistance to IMiD therapy in a subject with multiple myeloma undergoing IMiD therapy, the method comprising: a) measuring an amount of USP15 protein in the subject; b) comparing the amount of USP15 protein in the subject to a reference; c) diagnosing the subject with resistance to IMiD therapy if the amount of USP15 protein in the subject is higher than the reference; and d) administering a USP15 inhibitor to the subject.

A further aspect of the disclosure is directed to a method of regulating CRL4^{CRBN}-USP15 pathway and glutamine synthetase levels in a subject in need thereof, the method comprising: administering to the subject in need thereof a USP15 inhibitor.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In addition, the materials, methods, and examples are illustrative only and not intended to be limiting.

The details of one or more embodiments of the invention are set forth in the accompanying drawings and the description below. Other features, objects, and advantages will be apparent from the description and drawings, and from the claims.

### BRIEF DESCRIPTION OF THE DRAWINGS

FIG. 1A depicts a Western blot. 293FT cells were transfected with GS^{Myc} and USP15^{Flag}. After 48 h, cell extracts were immunoprecipitated with anti-Flag antibody.

FIG. 1B depicts a Western blot. Cell extracts from 293 cells were immunoprecipitated with rabbit IgG control or USP15 antibodies, and then analyzed by immunoblotting (IB) with the indicated antibodies.

FIG. 1C depicts a Western blot. Cell extracts from H1299 cells were immunoprecipitated with rabbit IgG control or USP15 antibodies, and then analyzed by immunoblotting (IB) with the indicated antibodies.

FIG. 1D depicts a Western blot. MCF7 cells, starved of glutamine for 24 h, were treated with MG132 (10 μM) and/or 40 μM NSC632839, 20 μM PR-619 for 0.5 h, followed by 4 mM glutamine treatment for 2 h. Total ubiquitylated proteins were purified using TUBE2-agarose, and then analyzed by IB with indicated antibodies. The relative ratio of ubiquitylated GS, GS(Ub)n to input GS, normalized to that of glutamine-treated cells in lane 2, are shown.

FIG. 1E depicts a Western blot. MCF7 cells, starved of glutamine for 24 h, were treated with NSC632839 for 0.5 h, followed by 4 mM glutamine treatment for 10 h. Cell extracts were analyzed by IB. The relative ratio of GS:Actin is shown.

FIG. 1F depicts a Western blot. MCF7 cells, starved of glutamine for 24 h, were treated with PR-619 for 0.5 h, followed by 4 mM glutamine treatment for 10 h. Cell extracts were analyzed by IB. The relative ratio of GS:Actin is shown.

FIG. 1G depicts a Western blot. H1299 cells were treated with NSC632839 and 2 μM MLN4924 for 0.5 h, followed by 4 mM glutamine treatment for 8 h. Cell extracts were analyzed by IB. The relative ratio of GS:Actin is shown.

FIG. 1H depicts a Western blot. H1299 cells were treated with PR-619 and 2 μM MLN4924 for 0.5 h, followed by 4 mM glutamine treatment for 8 h. Cell extracts were analyzed by IB. The relative ratio of GS:Actin is shown.

FIG. 2A depicts a Western blot. USP15^{+/+} and USP15^{-/-} 293FT cells (triplicate) were cultured in 2 mM glutamine. Cell extracts were analyzed by IB. The relative ratio of GS:Actin is shown.

FIG. 2B depicts a Western blot. USP15^{+/+} and USP15^{-/-} 293FT cells (triplicate) were cultured in different glutamine concentrations. Cell extracts were analyzed by IB. The relative ratio of GS:Actin is shown.

FIG. 2C depicts a Western blot. USP15^{+/+} and USP15^{-/-} 293FT cells (triplicate) were cultured in CHX. Cell extracts were analyzed by IB. The relative ratio of GS:Actin is shown.

FIG. 2D depicts a Western blot demonstrating rescue of endogenous GS protein levels in USP15-KO cells. USP15^{+/+} and USP15^{-/-} 293FT cells were grown in complete medium, and then treated with 2 μM MLN4924 for 10 h. Cell extracts were analyzed by IB with the indicated antibodies. The relative ratio of GS:Actin is shown.

FIG. 2E depicts a Western blot demonstrating rescue of endogenous GS protein levels in USP15-KO cells. USP15^{+/+} and USP15^{-/-} 293FT cells were grown in complete medium, and then treated with 2 mM MSO for 10 h. Cell extracts were analyzed by IB with the indicated antibodies. The relative ratio of GS:Actin is shown.

FIG. 2F depicts a Western blot demonstrating rescue of endogenous GS protein levels in USP15-KO cells. USP15^{+/+} and USP15^{-/-} 293FT cells were staved of glutamine for 24 h, and then pre-treated with 2 μM MLN4924 or 2 mM MSO for 30 min, followed by 4 mM glutamine treatment for 8 h. Cell extracts were analyzed by IB with the indicated antibodies. The relative ratio of GS:Actin is shown.

FIG. 2G depicts a Western blot. 293 cells stably expressing Dox-inducible shRNA targeting USP15 clone 1 (USP15-shRNA TRIPZ_1) were induced with or without Dox (2 μg/ml) for 72 h. Cell extracts were analyzed by IB with the indicated antibodies. The relative ratio of GS:Actin is shown.

FIG. 2H depicts a Western blot. 293 cells stably expressing Dox-inducible shRNA targeting USP15 clone 1 (USP15-shRNA TRIPZ_1) were induced with or without Dox (2 μg/ml) for 72 h. Cells were also grown in different glutamine concentrations. Cell extracts were analyzed by IB with the indicated antibodies. The relative ratio of GS:Actin is shown.

FIG. 2I depicts a Western blot. 293 cells stably expressing Dox-inducible shRNA targeting USP15 clone 1 (USP15-shRNA TRIPZ_1) were induced with or without Dox (2 μg/ml) for 72 h. Cells were also starved of glutamine for 24 h, followed by 4 mM glutamine treatment. Cell extracts were analyzed by IB with the indicated antibodies. The relative ratio of GS:Actin is shown.

FIG. 2J depicts a Western blot showing validation of USP15-knockout (KO) 293FT cells via CRISPR/Cas9 gene editing. Wild-type (WT) 293FT cells and USP15-KO clones 1 and 2 were validated by Western blot. USP15-KO2 293FT cells were chosen for further analysis.

FIG. 2K depicts a Western blot showing. 293 cells stably expressing Dox-inducible shRNA targeting USP15 clone 1 (USP15-shRNA TRIPZ_1) were induced with or without Dox (2 μg/ml) for 72 h. Cells were also treated with cycloheximide (CHX, 100 μg/ml). Cell extracts were analyzed by IB with the indicated antibodies. The relative ratio of GS:Actin is shown.

FIG. 2L depicts the relative GS mRNA level as analyzed by quantitative RT-PCR.

FIG. 3A depicts a Western blot. Glutamine-starved USP15^{+/+} and USP15^{-/-} 293FT cells were treated with 10 μM CB-5083 for 30 min, followed by 4 mM glutamine treatment for 3 h. The relative ratio of ubiquitylated GS, GS(Ub)n to input GS is shown.

FIG. 3B depicts a Western blot. Cells, grown in medium with 0.5 mM glutamine, were pre-treated with 2 μM MLN4924 or 2 mM MSO for 0.5 h, followed by treatment with CB-5083 for 6 h. Total ubiquitylated proteins were purified using TUBE2-agarose. The relative ratio of ubiquitylated GS, GS(Ub)n to input GS is shown.

FIG. 3C depicts a Western blot. USP15^{-/-} 293FT cells were transfected with control plasmid, wild-type WT USP15^{Flag} or C298A-USP15^{Flag} mutant plasmid. After 48 h, cells were treated with CB-5083 for 6 h. Total ubiquitylated proteins were affinity-purified using TUBE2-agarose. The relative ratio of ubiquitylated GS, GS(Ub)n to input GS is shown.

FIG. 3D depicts an in vitro deubiquitylation assay. USP15^{-/-} 293FT cells, starved off glutamine for 24 h, were pre-treated with CB-5083 for 0.5 h, followed by 4 mM glutamine treatment for 4 h. Total ubiquitylated proteins were affinity-purified using TUBE2-agarose beads, which were subsequently treated with or without recombinant USP15 (rUSP15) protein at 37° C. for 1 h, and then analyzed by Western blot. (Ub)n, polyubiquitin.

FIG. 3E depicts quantification of cell viability. LN229 cells were transduced with the GIPZ lentiviral vectors that expressed either control (CT) shRNA or USP15 shRNAs clones 4 and 6 (shUSP15). An equal number of cells (1×10⁶ cells in 10-cm plates) were then shifted to medium without glutamine in the presence (+) or absence (−) of the GS inhibitor MSO (2 mM) for 5 days, after which cell viability was quantified by staining with 0.4% Trypan blue. Cell viability was normalized to MSO-untreated control (−), which was set to 100%. Error bars represent the SEM of triplicates (P<0.0001 by t test).

FIG. 3F is related to FIG. 3A. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 3G is related to FIG. 3B. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 3H is related to FIG. 3C. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 3I is related to FIG. 3D. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 3J depicts a Western blot. LN229 cells, stably expressing non-target shRNA lentivirus (control: CT) or GIPZ lentiviral shRNA targeting human USP15 clones 4+6, were maintained in complete DMEM medium with 2 mM glutamine. Protein extracts were analyzed by immunoblotting with the indicated antibodies.

FIG. 4A depicts a Western blot. USP15^{+/+} and USP15^{-/-} 293FT cells, transfected with IKZF3^{FM} (IKZF3 with a C-terminal Flag-Myc tag) plasmid for 48 h, were pre-treated with bortezomib (Bort, 1 μM) for 0.5 h, followed by lenalidomide (Len) treatment at 0 and 20 μM for 5 h. Total ubiquitylated proteins were affinity-purified using TUBE2-agarose. Bound fractions and cell lysates (input) were analyzed by IB with indicated antibodies. (Ub)n: polyubiquitin.

FIG. 4B depicts a Western blot. Cells were pre-treated with Bort for 0.5 h, followed by Len treatment at 0, 10 and 20 μM for 5 h. Total ubiquitylated proteins were affinity-purified using TUBE2-agarose. Bound fractions and cell lysates (input) were analyzed by IB with indicated antibodies. (Ub)n: polyubiquitin.

FIG. 4C depicts a Western blot. Cells were pre-treated with CB-5083 (10 μM) for 0.5 h, followed by treatment with Len at 0, 10 and 20 μM for 5 h. Total ubiquitylated proteins were affinity-purified using TUBE2-agarose. Bound fractions and cell lysates (input) were analyzed by IB with indicated antibodies. (Ub)n: polyubiquitin.

FIG. 4D depicts a Western blot. Cells were pre-treated with MG132 (20 μM) for 1 h, followed by treatment with CC-885 at 0, 1 and 10 μM for 3 h. Total ubiquitylated proteins were affinity-purified using TUBE2-agarose. Bound fractions and cell lysates (input) were analyzed by IB with indicated antibodies. (Ub)n: polyubiquitin.

FIG. 4E depicts a Western blot. Cells were pre-treated with MG132 (20 PM) for 1 h, followed by treatment with dBET1, a potent BRD4 degrader (2 μM) for 4 h. Total ubiquitylated proteins were affinity-purified using TUBE2-agarose. Bound fractions and cell lysates (input) were analyzed by IB with indicated antibodies. (Ub)n: polyubiquitin.

FIG. 4F depicts a Western blot. USP15^{+/+} and USP15^{-/-} 293FT cells were pre-treated with 10 μM Len for 0.5 h, followed by addition of CHX. Samples were harvested for IB analysis. The relative ratio of CK1α:Actin was shown.

FIG. 4G depicts a Western blot. USP15^{-/-} 293FT cells, pre-treated with 10 μM CB-5083, 20 μM MG132 and 2 μM MLN4924 for 0.5 h, were treated with 10 μM CC-885 for 3 h. Total ubiquitylated proteins were affinity-purified using TUBE2-agarose.

FIG. 4H depicts a Western blot related to FIG. 4A. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 4I depicts a Western blot related to FIG. 4B. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 4J depicts a Western blot related to FIG. 4C. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 4K depicts a Western blot related to FIG. 4D. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 4L depicts a Western blot related to FIG. 4E. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 4M depicts a Western blot related to FIG. 4F. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 5A depicts a Western blot. USP15^{-/-} 293FT cells, pre-treated with CB-5083 (10 μM) for 0.5 h, were induced with 20 μM lenalidomide for 5 h. Total ubiquitylated proteins were affinity-purified using TUBE2-agarose beads. Polyubiquitylated CK1α proteins, pulled down with TUBE2 resin, were kept on ice as control (lane 1) or treated with or without recombinant USP15 (rUSP15) protein at 37° C. for 0.5 h (lanes 2 & 3, respectively), followed by IB analysis. CK1α(Ub)n: polyubiquitylated CK1α.

FIG. 5B depicts a Western blot. USP15^{-/-} 293FT cells, pre-treated with CB-5083 for 0.5 h, were treated with CC-885 (10 μM) treatment for 4 h. Total polyubiquitylated proteins, pulled down with TUBE2 resin, were treated with or without recombinant USP15 (rUSP15) protein in the absence or presence of DUB inhibitors NEM (N-Ethylmaleimide; 20 mM) and PR-619 (100 μM) at 37° C. for 0.5 h, followed by SDS-PAGE analysis and immunoblotting with antibodies against GSPT1, ubiquitin and USP15. GSPT1(Ub)n: polyubiquitylated GSPT1. Arrow indicates a native form of GSPT1, which was released after deubiquitylation of polyubiquitylated GSPT1 by rUSP15.

FIG. 5C depicts a Western blot. USP15^{-/-} 293FT cells, pre-treated with CB-5083 for 0.5 h, were treated with CC-885 (10 μM) treatment for 4 h. Total polyubiquitylated proteins, pulled down with TUBE2 resin, were treated with or without recombinant USP15 (rUSP15) protein in the absence or presence of indicated concentrations of PR-619 at 37° C. for 0.5 h, followed by SDS-PAGE analysis and immunoblotting analysis. SE and LE: short and long exposures. GSPT1(Ub)n: polyubiquitylated GSPT1. Arrow indicates a native form of GSPT1, which was released after deubiquitylation of polyubiquitylated GSPT1 by rUSP15.

FIG. 5D depicts a Western blot. IKZF3^{FM} was purified from USP15^{-/-} 293FT cells. An in vitro competitive ubiquitylation/deubiquitylation of IKZF3^{FM} was carried out for 1 h at 30° C. in the presence or absence of E1, E2, ^{HA}Ub and recombinant CLR4^{CRBN} complex purified from insect cells in a final volume of 30 μl. Where indicated, Len (lenalidomide 50 μM) and recombinant USP15 (rUSP15) were added. Reactions were stopped by mixing with equal amount of 2×SDS sample buffer and analyzed by SDS-PAGE and immunoblotting with antibodies against Flag, USP15 and CRBN. (Ub)n indicates polyubiquitylation. The relative ratio of ubiquitylated IKZF3^{FM}, IKZF3^{FM} (Ub)n to input IKZF3^{FM} is shown.

FIG. 5E depicts a Western blot related to FIG. 5A. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 5F depicts a Western blot related to FIG. 5B. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 5G depicts a Western blot related to FIG. 5C. Total ubiquitinated proteins were affinity-purified using TUBE2-agarose. Bound fractions were analyzed by SDS-PAGE and immunoblotting with anti-ubiquitin antibody. (Ub)n: polyubiquitin.

FIG. 6A depicts a Western blot. USP15 was highly expressed in lenalidomide-resistant MM cell lines. Equal amounts of protein extracts from 3 lenalidomide (Len)-sensitive MM cell lines and 4 Len-resistant MM cell lines were analyzed by IB with the indicated antibodies. The relative ratios of USP15:Actin, CRBN:Actin and CUL4A:Actin, normalized to MM1S cells, are shown.

FIG. 6B depicts a Western blot. USP15-shRNA TRIPZ_2 RPMI8226 cells were induced with or without Dox (2 μg/ml) for 96 h. Cells were treated with 10 μM Len for 24 and 48 h.

FIG. 6C depicts a Western blot. RPMI8226 cells, stably expressing CT shRNA lentivirus (shCT) or GIPZ lentiviral shRNAs targeting human USP15 clones 4 or 6 (shUSP15_4 or 6), were treated with Len at 0, 1 and 10 μM for 48 h.

FIG. 6D depicts a Western blot. KMS-11 cells, stably expressing shCT or shUSP15_6, were treated with Len for 24 h. Cell extracts were analyzed by IB with the indicated antibodies.

FIG. 6E depicts a Western blot. OPM-1 cells (E), stably expressing shCT or shUSP15_6, were treated with Len for 24 h. Cell extracts were analyzed by IB with the indicated antibodies.

FIG. 6F depicts cell viability demonstrating how depletion of USP15 sensitizes MM cells to lenalidomide. RPMI8226 cells stably expressing Dox-inducible shRNA TRIPZ targeting USP15 clone 2 (USP15TRIPZ_2) were induced with or without Dox (2 μg/ml) for 3 days, and then treated with DMSO or 2, 10 μM Len for 5 days. Cell viability was quantified by staining with 0.4% Trypan blue. Cell viability was normalized to DMSO-treated control, which was set to 100%. Error bars represent the SEM of triplicates (P<0.00001 by t test).

FIG. 6G depicts total cell number after different treatment conditions. RPMI8226 cells were treated with DMSO (control: CT) or the DUB inhibitor PR-619 (1 μM) in the presence of lenalidomide (Len) at the indicated concentrations for 4 days. Cell viability was quantified by staining with 0.4% Trypan blue. Error bars represent the SEM of triplicates (P<0.003 by t test).

FIG. 6H depicts a Western blot demonstrating validation of USP15 depletion. MM.1S cells were transduced with control shRNA lentivirus (CT), or GIPZ lentiviral shRNA particles targeting human USP15 by IB. USP15shRNA clones 4 and 6 with 80% knockdown efficiency were chosen for further analysis. Cell extracts were analyzed by immunoblotting with the indicated antibodies.

FIG. 6I depicts a Western blot. MM.1S cells, stably expressing control shRNA lentivirus (shCT) or GIPZ lentiviral shRNA targeting human USP15 clone 4 (shUSP15_4), were treated with Len at 0, 0.1 and 1 μM for 6 h. Cell extracts were analyzed by immunoblotting with the indicated antibodies.

FIG. 6J depicts a Western blot. (C) U266 cells (Dox-inducible USP15-shRNA TRIPZ_2) were induced with or without Dox for 96 h. Cells were treated with Len at the indicated doses for 6 h. Cell extracts were analyzed by immunoblotting with the indicated antibodies.

FIG. 6K depicts cell viability for various treatment conditions. MM.1S cells, stably expressing control shRNA lentivirus (shCT) or GIPZ lentiviral shRNA targeting human USP15 clones 4 or 6 (shUSP15_4 or 6), were treated with DMSO or 2 μM Len for 5 days, after which cell viability was quantified by staining with 0.4% Trypan blue.

FIG. 6L depicts a Western blot. U266 cells, pre-treated with the DUB inhibitor PR-619 (2 μM) for 1 h, were treated with lenalidomide at 0, 2 and 10 μM for 6 h. Cell extracts were analyzed by SDS-PAGE and immunoblotting with indicated antibodies.

FIG. 7A depicts a Western blot. Glutamine-starved 293FT cells were treated 4 mM glutamine for 2 h. Cell lysates were immunoprecipitated with CT IgG or USP15 antibody, and analyzed by IB with antibodies against GS, phosphorylated serine (P-Ser-USP15) or phosphorylated threonine (P-Thr-USP15). The relative ratio of GS bound to USP15 was normalized to input GS. The relative ratio of phosphorylated serine USP15 to USP15 input was normalized to untreated cells.

FIG. 7B depicts a Western blot. 293FT cells, transfected with CT or USP15^{Flag} plasmid for 24 h, were grown in complete medium (C; 2 mM glutamine) or starved of glutamine for 24 h, then treated with Torin1 for 0.5 h, followed by 4 mM glutamine treatment for 1-2 h. Cell extracts were immunoprecipitated with anti-Flag. The relative ratios of phosphorylated serine USP15^{Flag} to total input USP15^{Flag} and endogenous GS interacted with USP15^{Flag} to input GS, normalized to that of untreated cells in lane 3, are shown.

FIG. 7C depicts a Western blot. mTOR-shRNA TRIPZ_3 293FT cells, were induced with Dox for 24 h, and then transfected with CT or USP15^{Flag} plasmid. After 48 h, cell extracts were immunoprecipitated with anti-Flag and analyzed by IB with anti-phosphorylated serine. The relative ratio of phosphorylated serine USP15^{Flag} to USP15^{Flag} input is shown.

FIG. 8A depicts a Western blot. Glutamine-starved H1299 cells, pre-treated with 250 nM Torin1 and 10 μM CB5083 for 0.5 h, were stimulated with 4 mM glutamine for 4 h. Cell extracts were analyzed by IB with the indicated antibodies. The relative ratio of GS:Actin is shown.

FIG. 8B depicts a Western blot. mTOR-shRNA TRIPZ_3 HEK293 cells were induced with or without Dox for 72 h. Cell extracts were analyzed by IB with the indicated antibodies. The relative ratio of GS:Actin is shown.

FIG. 8C depicts a Western blot. mTOR-shRNA TRIPZ_3 293FT cells were induced with Dox for 72 h, and maintained in different glutamine concentrations for 24 h. Cell extracts were analyzed by IB with the indicated antibodies. The relative ratio of GS:Actin is shown.

FIG. 8D depicts a Western blot. mTOR-shRNA TRIPZ_3 293FT cells were induced with Dox for 72 h, and maintained in different glutamine concentrations for 24 h. Cells were treated with CHX for the indicated times. Cell extracts were analyzed by IB with the indicated antibodies. The relative ratio of GS:Actin is shown.

FIG. 8E depicts a Western blot. mTOR-shRNA TRIPZ_3 293FT cells were induced with Dox for 72 h, and maintained in different glutamine concentrations for 24 h. GS mRNA levels were quantified by qRT-PCR. Cell extracts were analyzed by IB with the indicated antibodies. The relative ratio of GS:Actin is shown.

FIG. 8F depicts a Western blot. mTOR-shRNA TRIPZ_3 293FT cells were induced with Dox for 72 h, and maintained in different glutamine concentrations for 24 h. Cells were treated with different concentrations of MLN4924 for 14 h. Cell extracts were analyzed by IB with the indicated antibodies. The relative ratio of GS:Actin is shown.

FIG. 8G depicts a Western blot. mTOR-shRNA TRIPZ_3 293FT cells were induced with Dox for 72 h, and maintained in different glutamine concentrations for 24 h. Cells were treated with different concentrations of MSO (G) for 14 h. Cell extracts were analyzed by IB with the indicated antibodies. The relative ratio of GS:Actin is shown.

FIG. 9 depicts a schematic diagram of FL human USP15 protein. It contains DUSP domain, ubiquitin-like fold (UBL) and ubiquitin carboxyl-terminal hydrolase (UCH).

FIG. 10A depicts a mass spectrum of a USP15 peptide containing phosphorylated S229.

FIG. 10B depicts a putative phosphorylation site at serine 229, located in the linker between the UBL domain and UCH domain, is conserved among mammalian USP15 orthologs.

FIG. 10C depicts the phosphorylation peptide abundance calculated by phosphorylated peptides/(phosphorylated peptides+unmodified peptides)*100%. The abundance of S229 phosphorylation (P-S229) was calculated and represented as mean±SD; n=2 per group (p=0.01035 by t test).

FIG. 11 depicts a proposed model where USP15 is a key regulator of the CRL4^{CRBN}-USP15 pathway to control the stability of natural substrate GS and neo-substrates.

### DETAILED DESCRIPTION

In this disclosure, chemical, RNA interference (RNAi), CRISPR/Cas9 gene editing, in vivo and in vitro biochemical approaches show that USP15 is a key regulator of the CRL4^{CRBN}-USP15 pathway (historically referred to as the ‘CRL4^{CRBN}-p97 pathway’) to control the stability of glutamine synthetase (GS) and neo-substrates, including IKZF1/3, CK1a, RNF166, GSPT1 and BRD4 (a target of CRBN-based PROTAC dBET1), all of which are crucial drug targets in many cancers. USP15 antagonizes ubiquitylation of CRL4^{CRBN} target proteins, thereby preventing their degradation. Notably, USP15 is highly expressed in IMiD-resistant cell lines, and depletion of USP15 sensitizes these cells to lenalidomide.

USP15 protein expression can be used as a predictive biomarker of response or resistance to IMiD therapy in patients with MM and also advance the development of a class of drugs to degrade pathogenic proteins in cancer via molecular glue degraders (CRBN E3 ligase modulators: CELMoDs) and proteolysis-targeting chimaeras (PROTACs). Inhibition of USP15 represents a valuable therapeutic opportunity to potentiate IMiD/CELMoD and PROTAC therapies for the treatment of cancer (such as glioblastoma (GBM), MM, Myelodysplastic syndrome with deletion 5q (Del(5q)MDS) and acute myeloid leukemia (AML)) and other human diseases. These findings provide a rationale for the clinical developments of USP15 inhibitors in combination with IMiD/CELMoD and CRBN-based PROTAC therapies for the treatments of many types of cancer.

The USP15 inhibitor is selected from USP15 anti sense mRNA, USP15 siRNA, USP15 shRNA, USP15 miRNA, USP15 oligonucleotides and chemical inhibitors of USP15 such as PR-619. These USP15 inhibitors can further be used in combination with IMiDs (now called CELMoDs including thalidomide, lenalidomide, pomalidomide, CC-122, CC-220 and CC-885) or CRBN-based PROTACs (dBET1) to treat cancer.

It was further shown that mTORC1-mediated phosphorylation of USP15 at serine 229 (P-USP15) results in increased USP15-GS interaction.

The present disclosure is directed to methods of treating cancer comprising administering a therapeutically effective amount of a USP15 inhibitor and at least one chemotherapeutic drug to a subject in need thereof.

The chemotherapeutic drug can be an immunomodulatory drug (IMiD), cereblon E3 ligase modulator (CELMoD), or CRBN-based proteolysis-targeting chimaera (PROTAC). The chemotherapeutic drug can be selected from the group consisting of thalidomide, lenalidomide, pomalidomide, CC-122, CC-220, and CC-885. The CRBN-based PROTAC can be selected from the group consisting of ARV-825 (2-((S)-4-(4-chlorophenyl)-2,3,9-trimethyl-6H-thieno[3,2-f][1,2,4]triazolo[4,3-a][1,4]diazepin-6-yl)-N-(4-(2-(2-(2-(2-((2-(2,6-dioxopiperidin-3-yl)-1,3-dioxoisoindolin-4-yl)amino)ethoxy)ethoxy)ethoxy)ethoxy)phenyl)acetamide), dBET1 ((6S)-4-(4-Chlorophenyl)-N-[4-[[2-[[2-(2,6-dioxo-3-piperidinyl)-2,3-dihydro-1,3-dioxo-1H-isoindol-4-yl]oxy]acetyl]amino]butyl]-2,3,9-trimethyl-6H-thieno[3,2-f][1,2,4]triazolo[4,3-a][1,4]diazepine-6-acetamide), dBRD9 (2-[[[4-(1,2-Dihydro-2-methyl-1-oxo-2,7-naphthyridin-4-yl)-2-6-dimethoxyphenyl]methyl]methylamino]-N-[2-[2-[2-[[2-(2,6-dioxo-3-piperidinyl)-2,3-dihydro-1,3-dioxo-1H-isoindol-4-yl]amino]ethoxy]ethoxy]ethyl]acetamide dihydrochloride), THAL-SNS-032 (N-(5-(((5-(tert-Butyl)oxazol-2-yl)methyl)thio)thiazol-2-yl)-1-(14-((2-(2,6-dioxopiperidin-3-yl)-1,3-dioxoisoindolin-4-yl)amino)-2-oxo-6,9,12-trioxa-3-azatetradecyl)piperidine-4-carboxamide), BJS-03-123 (N-[2-[2-[2-[2-[4-[6-[(6-Acetyl-8-cyclopentyl-7,8-dihydro-5-methyl-7-oxopyrido[2,3-d]pyrimidin-2-yl)amino]-3-pyridinyl]-1-piperazinyl]ethoxy]ethoxy]ethoxy]ethyl]-2-[[2-(2,6-dioxo-3-piperidinyl)-2,3-dihydro-1,3-dioxo-1H-isoindol-4-yl]oxy]acetamide), BSJ-02-162 (N-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)-2-((2-(2,6-dioxopiperidin-3-yl)-1,3-dioxoisoindolin-4-yl)amino)acetamide), BSJ-01-187 (7-cyclopentyl-2-((5-(4-(4-(2-((2-(2,6-dioxopiperidin-3-yl)-1,3-dioxoisoindolin-4-yl)amino)acetamido)butyl)piperazin-1-yl)pyridin-2-yl)amino)-N,N-dimethyl-7H-pyrrolo[2,3-d]pyrimidine-6-carboxamide), YKL-06-102 (2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-(2-(2-(2-(2-((2-(2,6-dioxopiperidin-3-yl)-1,3-dioxoisoindolin-4-yl)amino)ethoxy)ethoxy)ethoxy)ethyl)acetamide), BETd-246 (4-((3-Cyclopropyl-1-ethyl-1H-pyrazol-5-yl)amino)-7-(3,5-dimethylisoxazol-4-yl)-N-(3-(2-(2-(3-((2-(2,6-dioxopiperidin-3-yl)-1,3-dioxoisoindolin-4-yl)amino)propoxy)ethoxy)ethoxy)propyl)-6-methoxy-9H-pyrimido[4,5-b]indole-2-carboxamide), TL13-149 (N-(2-(2-(2-(2-((2-(4-(3-(5-(tert-butyl)isoxazol-3-yl)ureido)phenyl)benzo[d]imidazo[2,1-b]thiazol-6-yl)oxy)ethoxy)ethoxy)ethoxy)ethyl)-2-((2-(2,6-dioxopiperidin-3-yl)-1,3-dioxoisoindolin-4-yl)amino)acetamide), DD-04-015 (N-(2-(2-(2-(4-(6-((5-(3-(6-Cyclopropyl-8-fluoro-1-oxoisoquinolin-2(1H)-yl)-2-(hydroxymethyl)phenyl)-1-methyl-2-oxo-1,2-dihydropyridin-3-yl)amino)pyridin-3-yl)piperazin-1-yl)ethoxy)ethoxy)ethyl)-2-((2-(2,6-dioxopiperidin-3-yl)-1,3-dioxoisoindolin-4-yl)amino)acetamide), MT-802 (2-[2-[2-[4-[4-amino-3-(4-phenoxyphenyl)pyrazolo[3,4-d]pyrimidin-1-yl]piperidin-1-yl]ethoxy]ethoxy]-N-[2-(2,6-dioxopiperidin-3-yl)-1,3-dioxoisoindol-5-yl]acetamide), MS4078 (2-(4-(4-((5-Chloro-4-((2-(isopropylsulfonyl)phenyl)amino)pyrimidin-2-yl)amino)-5-isopropoxy-2-methylphenyl)piperidin-1-yl)-N-(2-((2-(2,6-dioxopiperidin-3-yl)-1,3-dioxoisoindolin-4-yl)amino)ethyl)acetamide), GSK983 ((R)—N-(6-chloro-2,3,4,9-tetrahydro-1H-carbazol-1-yl)picolinamide), MD-224 ((3′R,4'S,5′R)-6″-chloro-4′-(3-chloro-2-fluorophenyl)-N-(4-((5-(2-(2,6-dioxopiperidin-3-yl)-1-oxoisoindolin-4-yl)pent-4-yn-1-yl)carbamoyl)phenyl)-2″-oxodispiro[cyclohexane-1,2′-pyrrolidine-3′,3″-indoline]-5′-carboxamide), and L18I (3-(4-(3-(2-(2-(2-(4-((R)-3-(4-Amino-3-(4-phenoxyphenyl)-1H-pyrazolo[3,4-d]pyrimidin-1-yl)piperidine-1-carbonyl)-1H-1,2,3-triazol-1-yl)ethoxy)ethoxy)ethoxy)propyl)-1-oxoisoindolin-2-yl)piperidine-2,6-dione).

The cancer can be multiple myeloma, glioblastoma, deletion 5q subtype of myelodysplastic syndrome, or acute myeloblastic leukemia. The cancer can be IMiD-resistant.

The disclosure is further directed to methods of diagnosing and treating resistance to IMiD therapy in a subject with multiple myeloma undergoing IMiD therapy, the method comprising: a) measuring an amount of USP15 protein in the subject; b) comparing the amount of USP15 protein in the subject to a reference; c) diagnosing the subject with resistance to IMiD therapy if the amount of USP15 protein in the subject is higher than the reference; and d) administering a USP15 inhibitor to the subject. Measuring the amount of USP15 protein in the subject can comprise using immunohistochemical (IHC) staining of USP15 protein in bone marrow sections of the subject. The reference can be obtained from measuring an amount of USP15 protein from a different subject or subjects without multiple myeloma.

Further disclosed are methods of regulating CRL4^{CRBN}-USP15 pathway and glutamine synthetase levels in a subject comprising administering a USP15 inhibitor to the subject.

For any of the disclosed methods, the USP15 inhibitor can be a nucleic acid or a chemical inhibitor. The nucleic acid can be selected from the group consisting of USP15 antisense mRNA, USP15siRNA, USP15shRNA, USP15 miRNA, and USP15 oligonucleotides. The chemical inhibitor can be a deubiquitinating enzyme (DUB) inhibitor. The chemical inhibitor can be selected from the group consisting of PR-619, NSC632839, and N-Ethylmaleimide (NEM). The chemical inhibitor can be mitoxantrone or an ubiquitin variant.

For any of the disclosed methods, the subject can be a mammal. The subject can also be a human, mouse, or rat.

As used in this application, including the appended claims, the singular forms “a,” “an,” and “the” include plural references unless the content clearly dictates otherwise, and are used interchangeably with “at least one” and “one or more.”

The invention will be further described in the following examples, which do not limit the scope of the invention described in the claims.

### EXAMPLES

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

### Example 1: USP15 is a GS-Interacting Partner

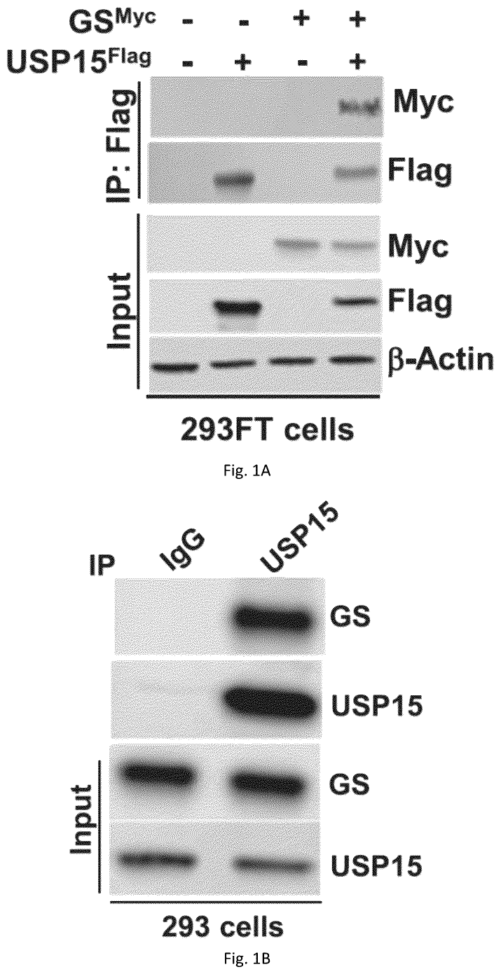

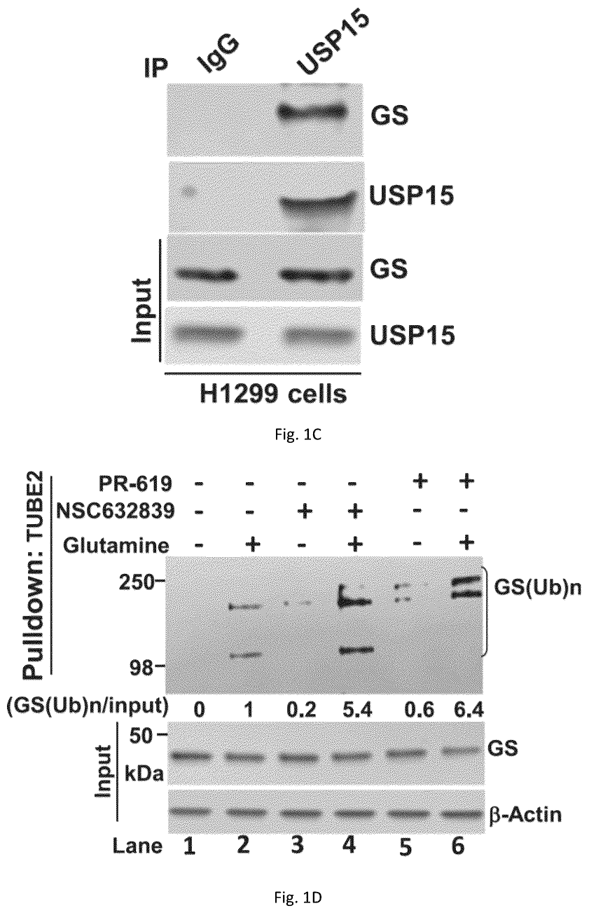

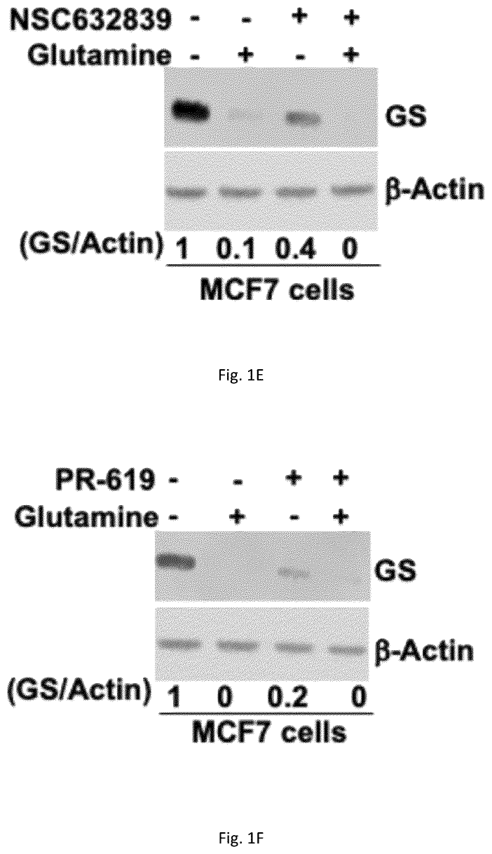

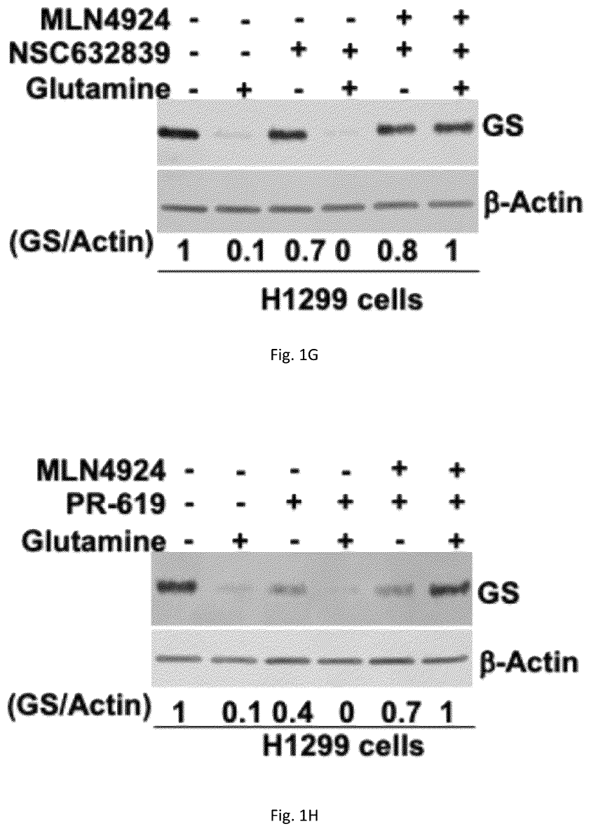

To identify participants in regulating GS degradation in the CRL4^{CRBN}-p97 pathway, GS-interacting proteins were sought. Previously, two independent mass spectrometry (MS) analyses of immunoprecipitation (IP) of endogenous USP15 prepared from LN-229 cells or two leukemia cell lines (MV-4-11 and Kasumi-1) revealed that GS was one of the top USP15-interacting proteins. To validate these proteomic data, immunoprecipitation experiments were used to find that USP15^{Flag} interacts with GS^{Myc} (FIG. 1A). Strikingly, endogenous GS interacted with endogenous USP15 in different cell lines (FIGS. 1B and 1C). These results demonstrated that USP15 is a GS-interacting partner.

Previous in-depth analysis of the ubiquitinome using stable isotope labeling of amino acids in cell culture (SILAC)-based quantitative MS from Jurkat cells treated with the proteasome inhibitor MG-132 or the DUB inhibitor PR-619 for 4 h revealed that while increased ubiquitylation of GS was observed in cells treated with MG132, PR-619 treatment decreased GS ubiquitylation, suggesting that GS is rapidly ubiquitylated and degraded in PR-619-treated cells, leading to a decrease in abundance of both native and ubiquitylated forms of GS. To test this possibility, the effects of two DUB inhibitors NSC632839 and PR-619 on GS ubiquitylation and abundance were evaluated. Consistent with this proteomic study, inhibition of DUB activity by NSC632839 or PR-619 promoted glutamine-induced ubiquitylation of GS (FIG. 1D). In addition, NSC632839 and PR-619 treatment resulted in decreased protein levels of endogenous GS (FIG. 1E-F). Next it was tested whether the action of DUB inhibitors on acceleration of glutamine-induced GS degradation is indeed dependent on the E3 ubiquitin ligase CRL4^{CRBN}. Cells were co-treated with NSC632839 or PR-619 and the NEDD8-activating enzyme inhibitor MLN4924 and found that MLN4924 blocked the action of DUB inhibitors on glutamine-induced GS degradation (FIG. 1G-H). Together, these results indicate that USP15 regulates GS ubiquitylation and degradation in an E3 ligase activity-dependent manner.

### Example 2: Depletion of USP15 Induces GS Degradation

Next, the molecular mechanism by which USP15 might control GS stability via the CRL4^{CRBN}-p97 pathway was studied using RNA interference (RNAi) and CRISPR/Cas9 gene editing approaches. A previous study using stable isotope labeling of amino acids in cell culture (SILAC)-based quantitative MS to characterize changes in the ubiquitinome of Jurkat cells treated with the proteasome inhibitor MG-132 or the DUB inhibitor PR-619 for 4 h revealed that while increased ubiquitylation of GS was observed in cells treated with MG132, PR-619 treatment decreased the ubiquitination of GS, suggesting that GS is rapidly ubiquitylated and degraded in PR-619-treated cells, leading to a decrease in abundance of both native and ubiquitylated forms of GS.

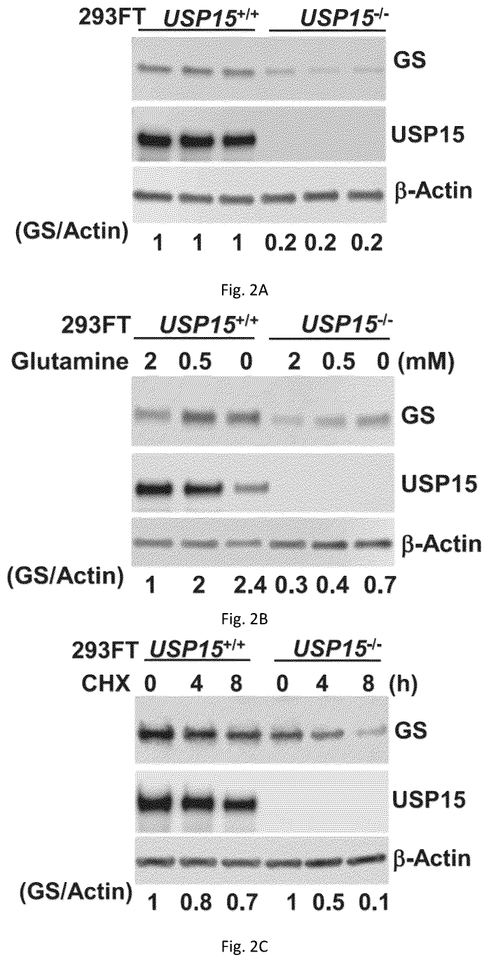

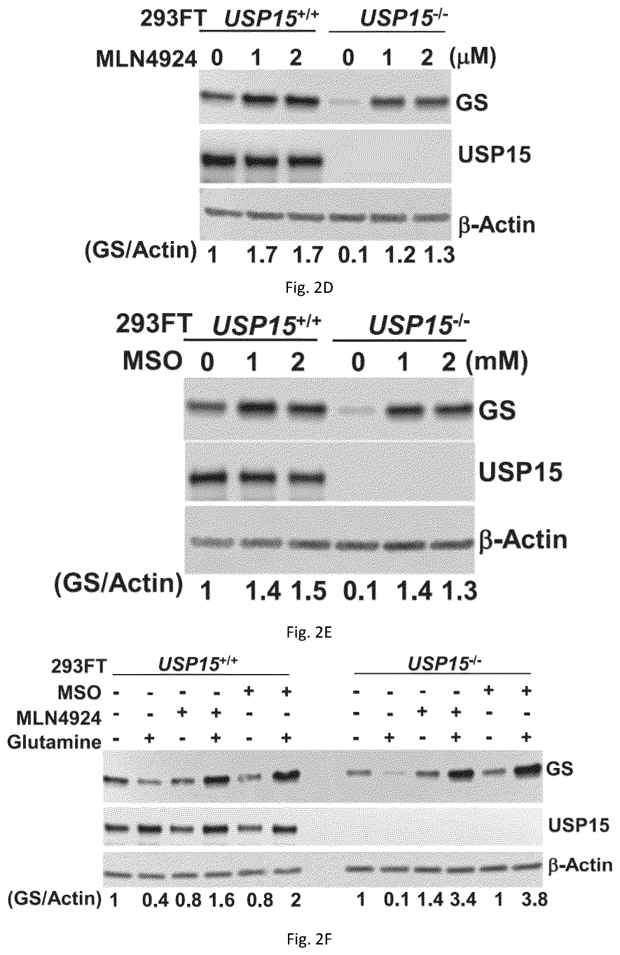

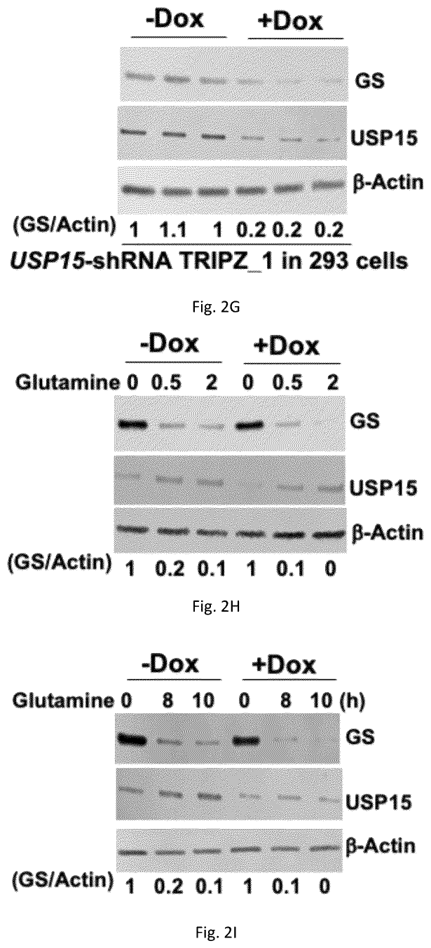

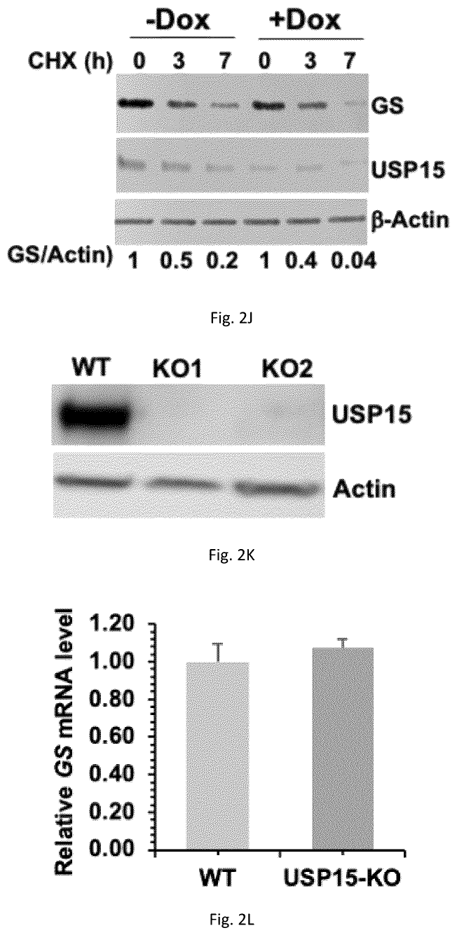

First the USP15-GS interaction was disrupted by depletion of USP15 using doxycycline (Dox)-inducible shRNA in TRIPZ system. Consistent with the results obtained with the DUB inhibitors, a similar result was obtained upon shRNA knockdown of USP15. Depletion of USP15 by shRNA induced GS degradation (FIG. 2G-2J). Most importantly, the steady-state level of GS was significantly downregulated in USP15-knockout (KO) 293FT cells generated by CRISPR/CAS9 (FIGS. 2A, 2B and 2K). The CHX chase experiments revealed that GS protein was markedly degraded in USP15-KO cells (FIG. 2C). The USP15-dependent degradation of GS is a post-translational regulation because the mRNA level of GS remained unchanged in USP15-KO 293FT cells (FIG. 2L). As shown in FIG. 2D-2F, the downregulation of endogenous GS protein levels observed in USP15-KO 293FT cells was rescued by blocking GS ubiquitylation using MLN4924 or the GS inhibitor MSO in a prior study, indicating that USP15 controls GS degradation in downstream of CRL4^{CRBN}.

### Example 3: USP15 Deubiquitylates Polyubiquitylated GS In Vivo and In Vitro

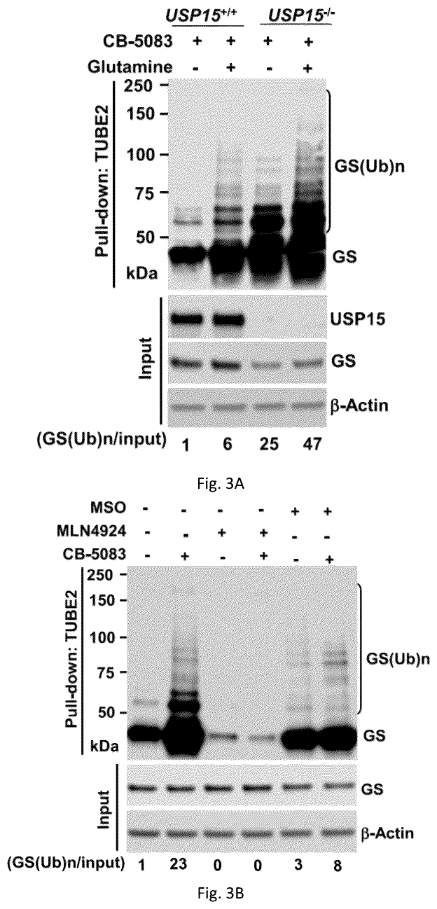

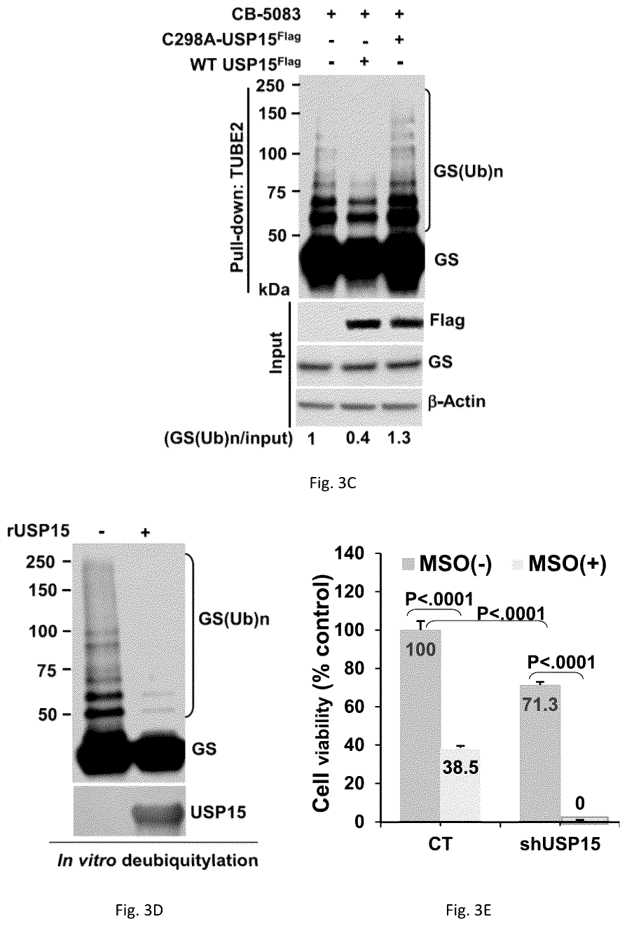

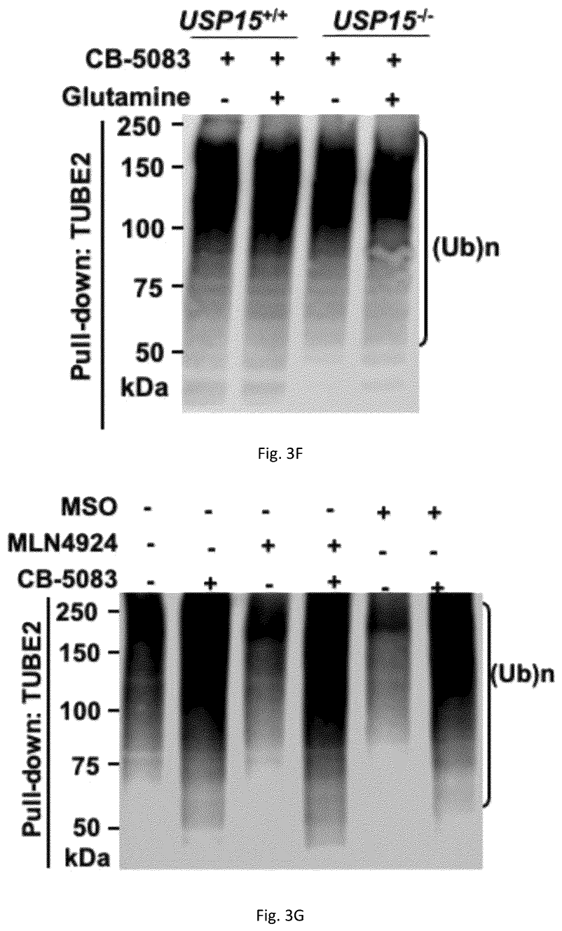

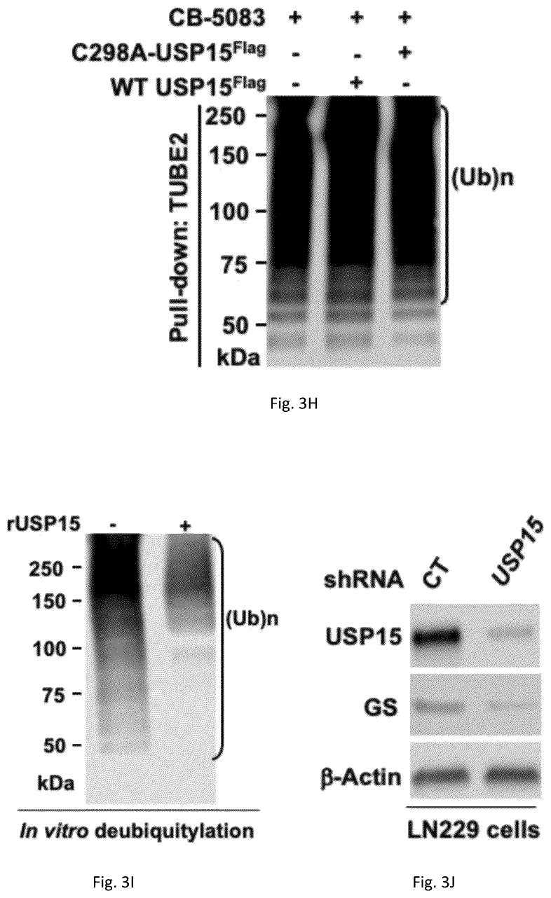

Since degradation of GS is dependent on the UPS, it was next sought to examine the effect of USP15 depletion on GS ubiquitylation. Indeed, a significant increase in ubiquitylated GS forms was detected in USP15^{-/-} 293FT cells (FIGS. 3A and 3F). To further define the molecular events in which USP15 participated in regulation of GS ubiquitylation, TUBE2 pulldown experiments were performed using USP15^{-/-} 293FT cells, grown in complete medium with 0.5 mM glutamine (the same glutamine concentration in human plasma), and it was found that the accumulation of ubiquitylated GS was observed in cells treated with the p97 inhibitor CB-5083 (FIGS. 3B and 3G, lane 2), but not in cells co-treated with MLN4924 and MSO, which were recently reported to inhibit GS ubiquitylation, suggesting that USP15 controls GS ubiquitylation by acting in downstream of CRL4^{CRBN} and upstream of p97, possibly dependent on its DUB activity. Consistent with this notion, overexpression of WT USP15^{Flag} abrogated polyubiquitylated GS, while a catalytically inactive C298A-USP15^{Flag} mutant failed to deubiquitylate polyubiquitylated GS in USP15^{-/-}293FT (FIGS. 3C and 3H). Most importantly, recombinant USP15 enzyme (rUSP15) purified from insect cells completely deconjugated polyubiquitylated GS in vitro (FIGS. 3D and 3I). These findings strongly indicate that USP15 is a component of the CRL4^{CRBN}-p97 pathway, and that it deubiquitylates poly-ubiquitylated GS in vivo and in vitro and stabilizes GS protein levels in cells.

GBM is the most frequent adult primary malignant brain tumors, and it is the second leading cause of cancer mortality in adults under 35 years of age. GBM and many other tumors expressing high GS levels can synthesize glutamine de novo, grow and proliferate in the absence of exogenous glutamine. Interestingly, USP15 amplification confers poor prognosis in patients with GBM. The above data provide compelling evidence that USP15 is a key component of CRL4^{CRBN}-p97 pathway to regulate GS stability. It was found that depletion of USP15 by shRNAs in LN229 cells resulted in a marked reduction in the steady-state level of GS (FIG. 3J). Depletion of USP15 by shRNAs or inhibition of GS by the GS inhibitor MSO in control LN229 cells exhibited a severe defect in cell viability (FIG. 3E). In line with previous research, these results confirm the important role of the USP15-GS axis in maintaining GBM cell viability. Strikingly, depletion of both USP15 by shRNAs and GS by MSO produced synergistic inhibition of cell viability in LN229 cells (FIG. 3E). Taken together, these results indicate the importance of USP15 in the regulation of glutamine metabolism via GS enzyme that is essential for tumor growth.

### Example 4: USP15 Regulates CELMoD-Induced Ubiquitylation and Degradation of Neo-Substrates

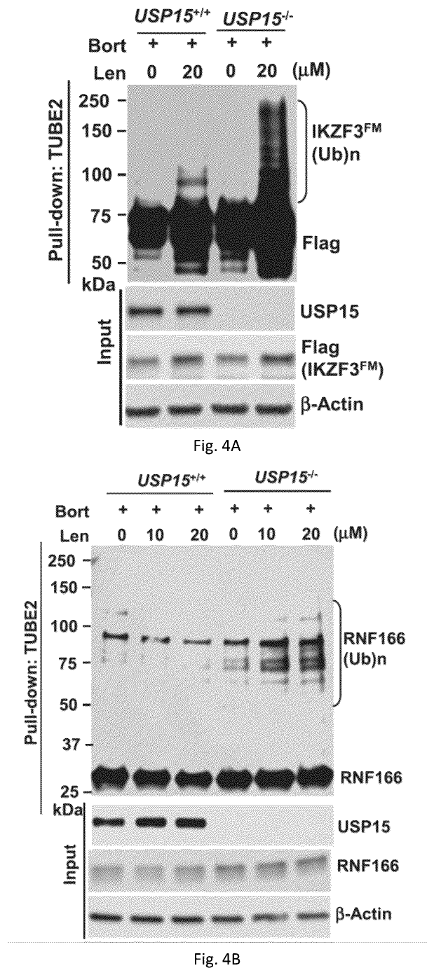

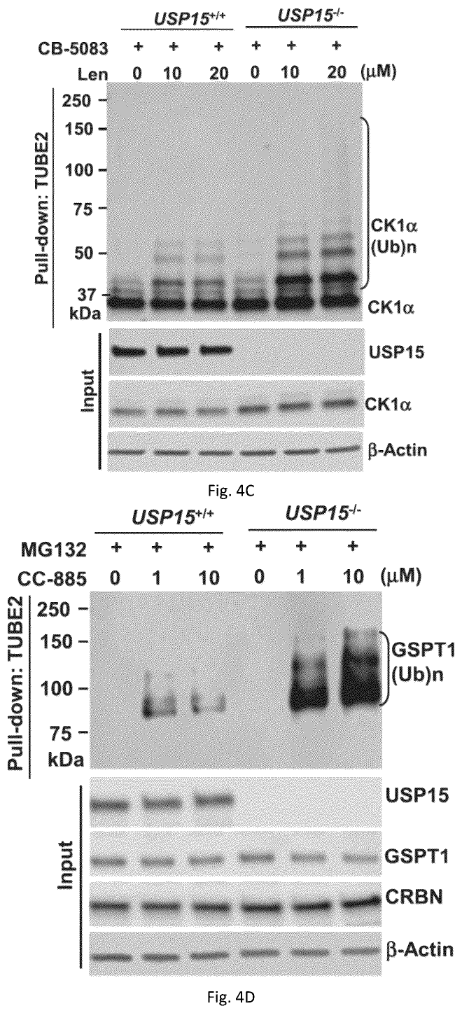

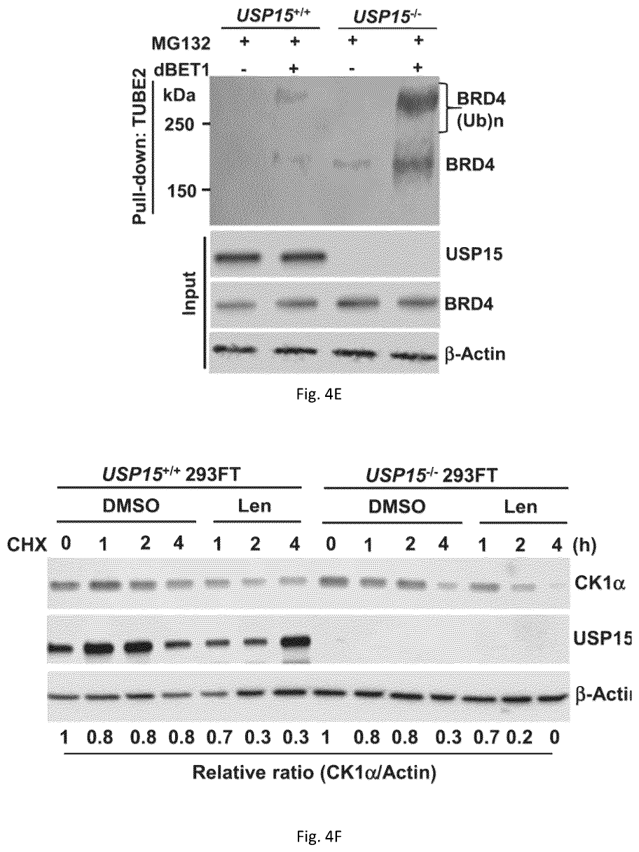

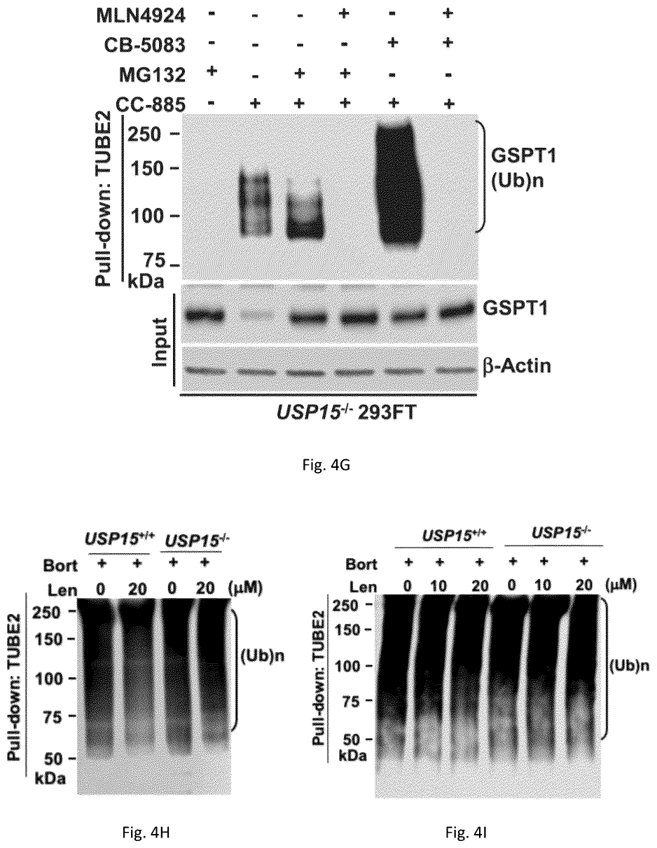

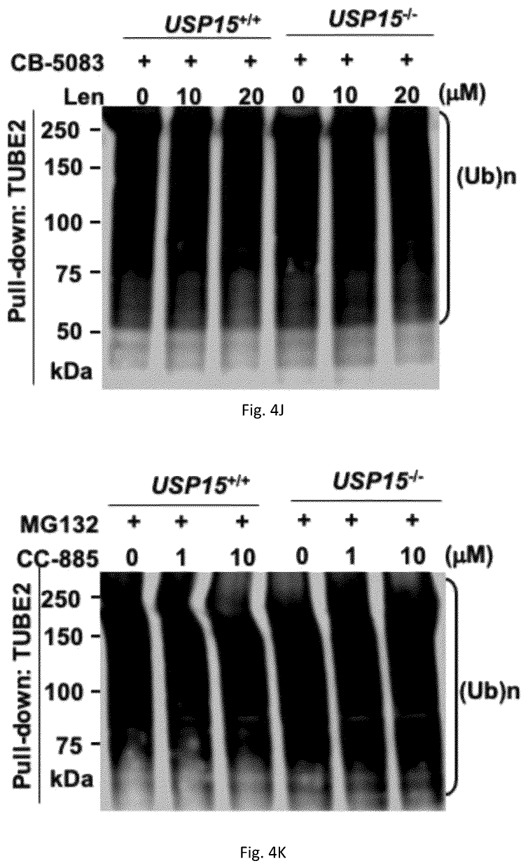

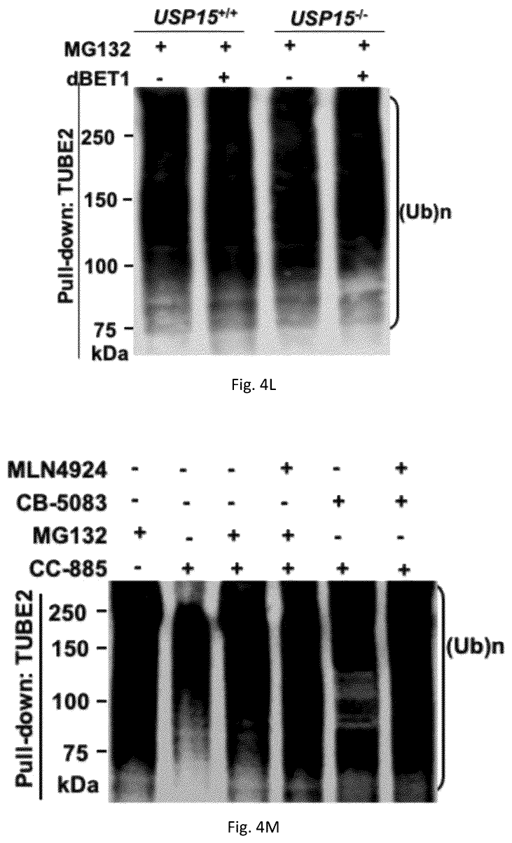

Several groups reported that the molecular glue degrader CELMoDs and the PROTAC degrader dBET1 degrade CRL4^{CRBN} neo-substrates or target proteins via molecular glue and PROTAC mechanisms, respectively. Notably, the CRL4^{CRBN}-p97 pathway was uncovered, which is required for degradation of both endogenous substrate GS and neo-substrates of CRL4^{CRBN}. It was next sought to investigate a potential role for USP15 in regulating ubiquitylation of CRL4^{CRBN} neo-substrates. As shown in FIGS. 4A-E and 4H-L, lenalidomide, CC-885 and dBET1 promoted accumulation of polyubiquitylated forms of all tested neo-substrates including IKZF3, CK1α, RNF166, GSPT1 and BRD4 in USP15-KO 293FT cells, suggesting that USP15 plays an important role in regulating ubiquitylation status of these target proteins, possibly through its deubiquitylating enzyme activity. It was further tested if depletion of USP15 could have the effects on CELMoD-induced degradation of neo-substrates. CHX chase experiments were performed to monitor lenalidomide-induced degradation of CK1α and found that lenalidomide significantly enhanced degradation of CK1α in USP15-KO 293FT cells (FIG. 4F). To further define the sequential events in which USP15 controls CELMoD-induced ubiquitylation and degradation of neo-substrates, TUBE2 pulldown was performed using cell extracts of USP15^{-/-} 293FT cells treated with different inhibitors and observed that polyubiquitylated GSPT1 was accumulated in the cells co-treated with CC-885 and MG132 or CB-5083, which was completely abolished in the presence of MLN4924 (FIGS. 4G and 4M). In addition, CC-885-induced degradation of GSPT1 was blocked in cells co-treated with CC-885 and MG132 or CB-5083 (FIG. 4G, lower panel). These results indicated that USP15 functions downstream of CRL4^{CRBN} and upstream of p97 and the proteasome to control the stability of all neo-substrates.

### Example 5: USP15 Deubiquitylates Polyubiquitylated Neo-Substrates In Vitro

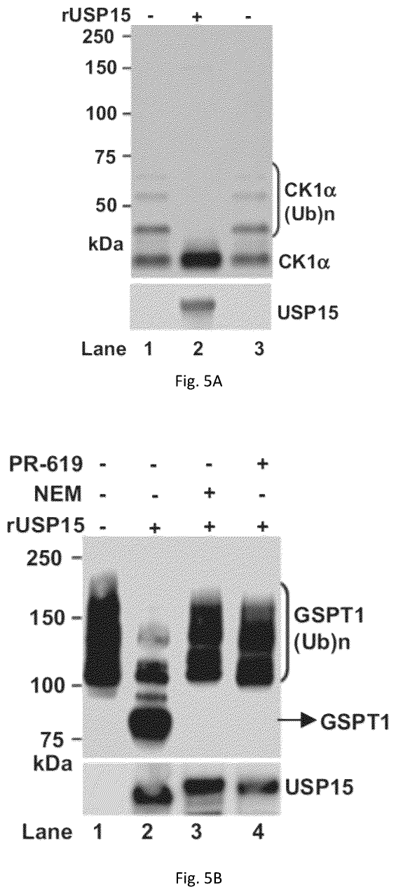

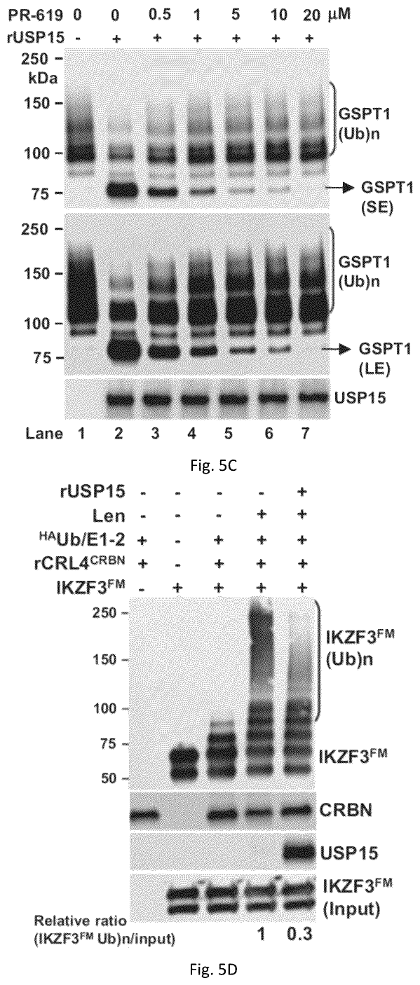

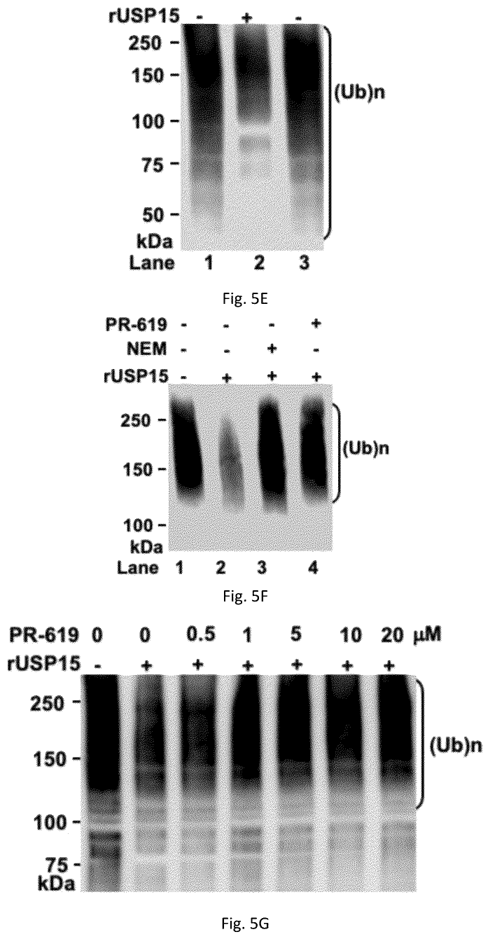

Since USP15 is a deubiquitylating enzyme, it was next tested whether it could remove polyubiquitin chains from neo-substrates of CRL4^{CRBN} in vitro. To achieve this goal, ubiquitin-binding TUBE2 resin was utilized to purify polyubiquitylated neo-substrates from cellular extracts of USP15-KO cells treated with the CELMoDs lenalidomide and CC-885 in the presence of p97 or proteasome inhibitor, and then performed the in vitro deubiquitylation assays using rUSP15. USP15 directly deubiquitylates polyubiquitylated CK1α forms (FIGS. 5A and 5E) and GSTP1 (FIGS. 5B and 5F). Strikingly, the activity of USP15 was completely inhibited in the presence of the broad-spectrum DUB inhibitors N-Ethylmaleimide (NEM) and PR-619 at a high dose of 100 μM (FIGS. 5B and 5F). This was consistent with a previous study, showing that PR-619 can inhibit the activity of almost all cysteine protease DUBs, including USP15. It was further demonstrated that PR-619 with the concentration range of 1-20 μM substantially suppressed USP15 activity in a dose-dependent manner (FIGS. 5C and 5G). It was next determined whether USP15 might directly antagonize CRL4^{CRBN}-mediated ubiquitylation of IKZF1/3 in vitro. To this end, the ‘competitive’ ubiquitylation/de-ubiquitylation assay that contains all key components of the UPS, including recombinant E1, E2, CRL4^{CRBN} neo-substrate IKZF3^{FM} (FM: C-terminal Flag-Myc) purified from USP15^{-/-} 293FT cells and rUSP15 was performed in the presence or absence of lenalidomide to mimic the biological properties of in vivo cells. The in vitro competitive ubiquitylation/deubiquitylation assay strongly indicated that lenalidomide promoted CRL4^{CRBN}-mediated ubiquitylation of IKZF3 in vitro in an IMiD-dependent manner (FIG. 5D), consistent with two previous studies (9,10). In striking contrast, lenalidomide-induced ubiquitylation of IKZF3 was significantly inhibited in the presence of rUSP15 (FIG. 5D). Taken together, these results demonstrated that USP15 is a regulator of the CRL4^{CRBN}-p97 pathway to control CELMoD-induced ubiquitylation and subsequent degradation of neo-substrates.

### Example 6: Depletion of USP15 Promotes IMiD-Induced Degradation of IKZF1/3 in Lenalidomide-Resistant MM Cell Lines

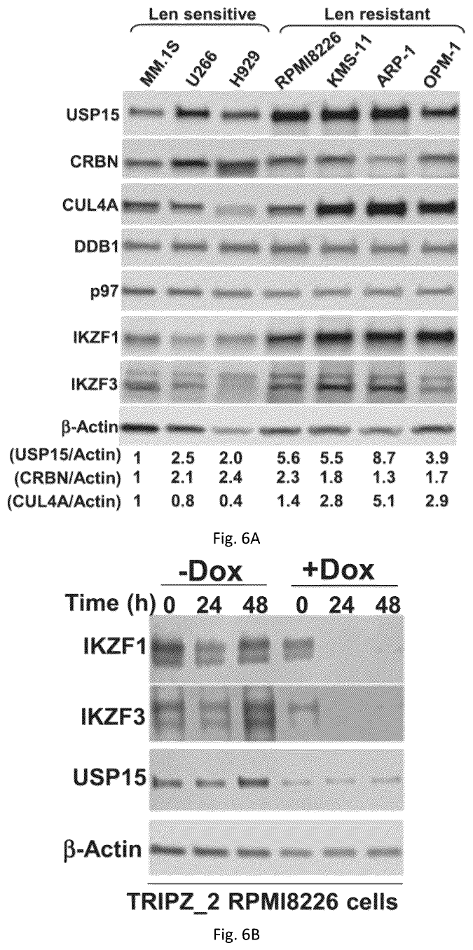

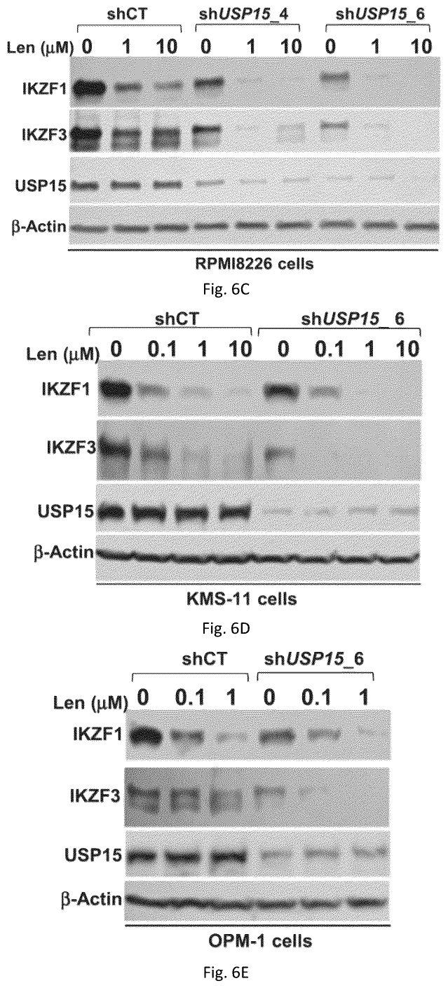

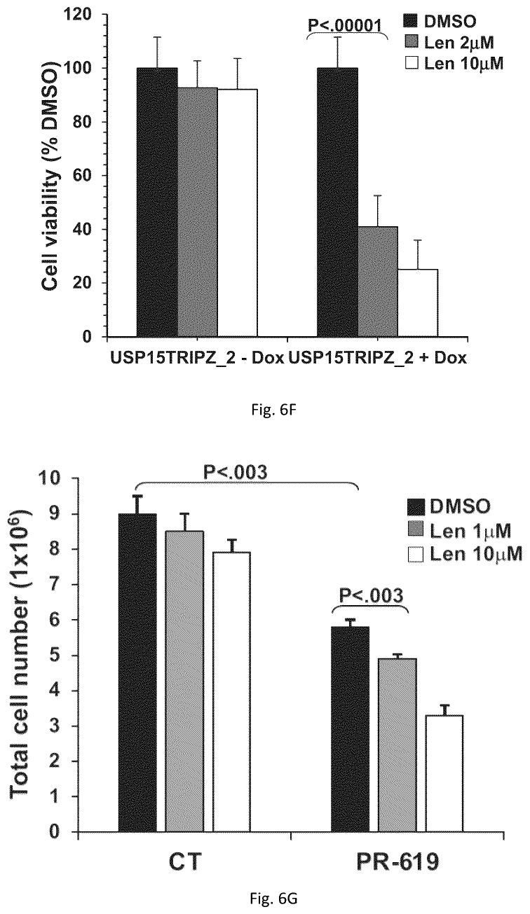

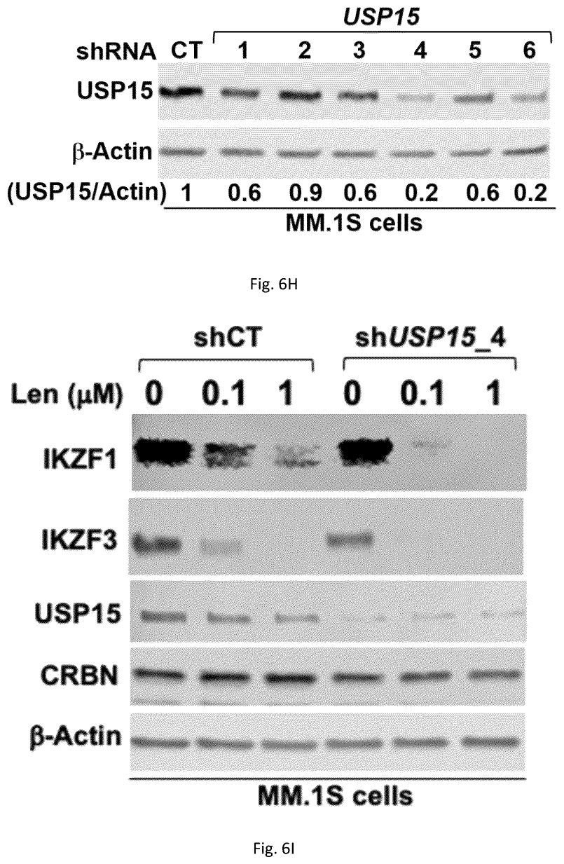

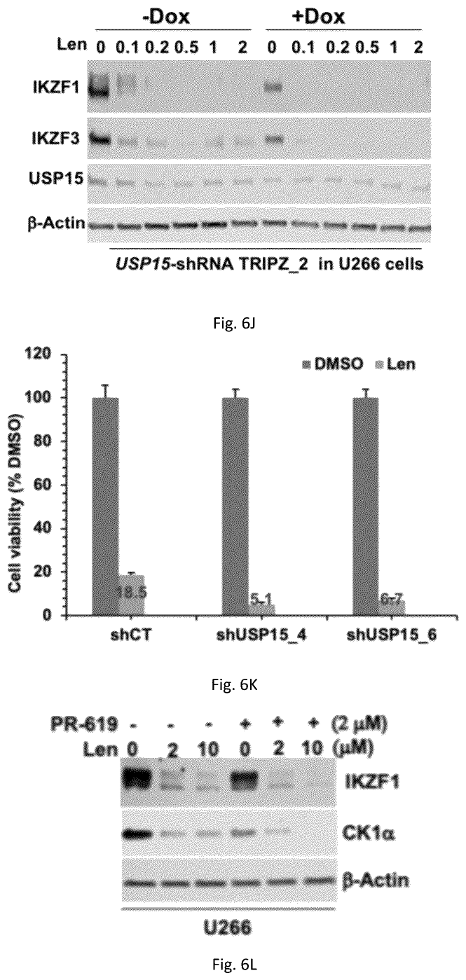

To gain further insight into the molecular mechanisms underlying IMiD sensitivity and resistance in MM, the USP15 protein levels were analyzed in lenalidomide (Len)-sensitive and Len-resistant MM cell lines by immunoblotting with antibodies against USP15 and core components of the CRL4^{CRBN}-p97 pathway. While the protein levels of p97 and DDB1 remained unchanged in all tested lenalidomide-sensitive and resistant cell lines, the protein levels of CRBN and CUL4A in Len-resistant cell lines expressed at least equal or higher, compared with those in Len-sensitive cell lines (FIG. 6A). In contrast, the protein levels of USP15 in all Len-resistant cell lines were substantially overexpressed compared to the levels observed in Len-sensitive cell lines (FIG. 6A), indicating that USP15 plays an important role in IMiD resistance. Consistent with the role of USP15 in regulating IMiD-induced ubiquitylation and degradation of neo-substrates in USP15^{-/-} 293FT cells (FIG. 4A-4F), knockdown of USP15 in the len-resistant RPMI8226 cell line by 2 different RNAi approaches, including USP15-shRNA TRIPZ_2 (FIG. 6B) or GIPZ lentiviral shRNAs targeting human USP15 clones 4 or 6 (shUSP15_4 or 6) (FIG. 6C) promoted IMiD-induced degradation of IKZF1/3 proteins. Consistent results were observed in two other Len-resistant cell lines KMS-11 (FIG. 6D) and OPM-1 (FIG. 6E). USP15 also contributed to the antitumor activities of IMiDs since depletion of USP15 also enhanced IKZF1/3 degradation in two different IMiD-sensitive MM cell lines MM1.S (FIG. 6I) and U266 (FIG. 6J) These findings indicated that USP15 counteracted IMiD-induced ubiquitylation of CRL4^{CRBN} neo-substrates including IKZF1/3 in MM cells, thus preventing their degradation. Depletion of USP15 sensitizes MM.1S cells (FIG. 6K) and RPMI8226 to lenalidomide (FIG. 6F). Currently, there is no inhibitor available to specifically inhibit USP15. However, multiple lines of evidence from the above data indicate that PR-619, a broad-spectrum DUB inhibitor, inhibits USP15 in vivo (FIG. 1D) and in vitro (FIG. 5B-5C). Co-treatment of U266 cells with PR-619 and lenalidomide significantly promoted degradation of neo-substrates (FIG. 6L). Inhibition of USP15 with PR-619 at the lowest dose (1 μM) sensitized resistant cell line RPMI8226 to lenalidomide (FIG. 6G).

### Example 7: Glutamine Activates mTORC1-Dependent Phosphorylation of USP15

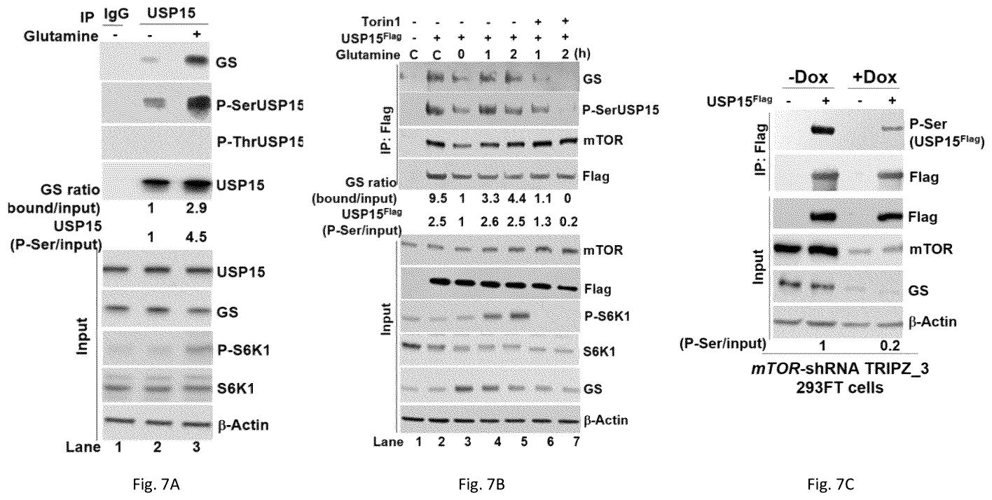

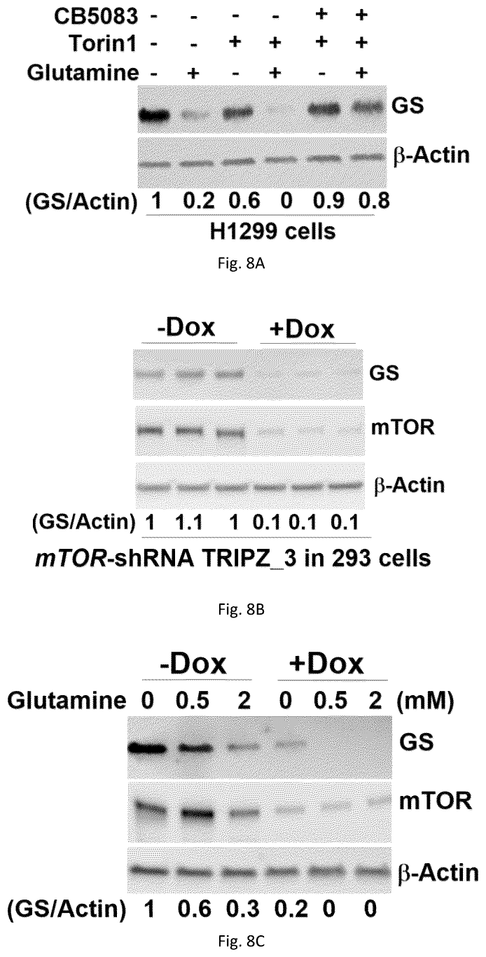

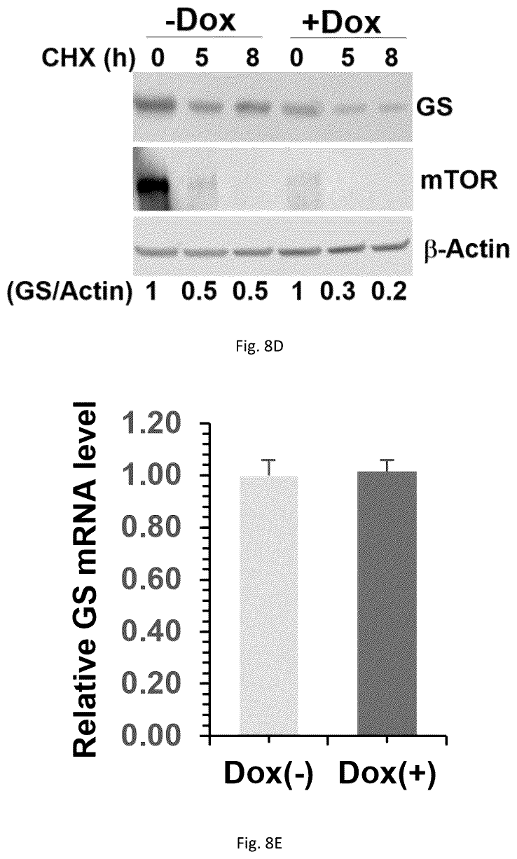

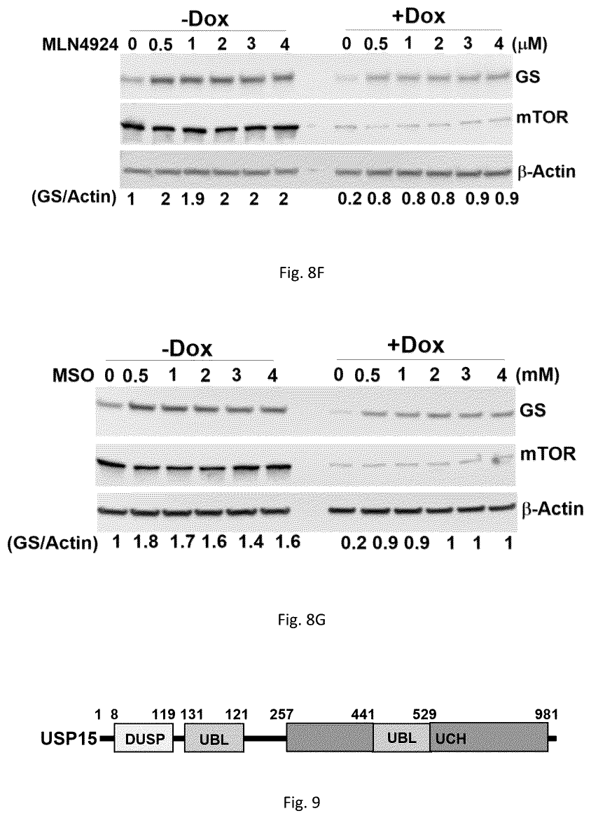

Glutamine activates the highly conserved, atypical Serine/Threonine kinase mammalian Target of Rapamycin Complex 1 (mTORC1), which regulates protein translation, cell growth and autophagy. The core components of mTORC1 consist of mTOR, Raptor, and mLST8. Glutamine may directly activate USP15 through mTORC1. IP experiments were used to determine that glutamine significantly stimulated the binding of endogenous GS to endogenous USP15, and correlated with enhanced serine-phosphorylated, but not threonine-phosphorylated USP15 (FIG. 7A). Elevated mTORC1 activity was also observed in glutamine-stimulated cells, exhibiting increased phosphorylation of S6K1 (P-S6K1) (FIG. 7A). These results indicated that mTORC1 senses glutamine levels and activated USP15 to control GS stability. To investigate this, USP15Flag was first overexpressed in 293FT cells, and the cells were cultured under various conditions. The IP experiments showed that glutamine-induced phosphorylated serine USP15^{Flag} (P-Ser-USP15) was substantially abolished in mTORC1-depleted cells by Torin-1 (FIG. 7B). A significant reduction in binding of USP15 to GS was also observed in Torin1-treated cells (FIG. 7B), indicating that mTORC1-mediated phosphorylation of USP15 was critical for the USP15-GS interaction. Interestingly, USP15 was a mTOR-interacting partner (FIG. 7B). Consistently, depletion of mTOR by shRNA significantly reduced accumulation of USP15 phosphorylation (FIG. 7C). In addition, endogenous protein level of GS was markedly downregulated in mTOR-depleted cells (FIG. 7C). Next, loss-of-function studies were performed to examine the role of mTORC1 in regulating GS stability. First glutamine-starved H1299 cells were co-treated with a potent mTOR inhibitor Torin-1 and the p97 inhibitor CB5083, and then stimulated cells with glutamine. As shown in FIG. 8A, inhibition of mTOR promoted glutamine-induced GS degradation, which was blunted by CB5083, indicating that mTOR functions downstream of p97 to regulate GS stability. Consistent with these results, a recent study reported that mTOR regulates GS degradation in a proteasome-dependent manner through an unknown mechanism. To further determine the role of mTORC1 in GS degradation, Dox-inducible shRNA was used in TRIPZ system to deplete mTOR in HEK293 and 293FT cells. Depletion of mTOR by shRNA promoted GS degradation (FIG. 8B-8D) while the mRNA levels of GS remained unchanged in mTOR knockdown cells (FIG. 8E). Since mTOR functions the same axis as USP15, its action in mediating GS degradation is downstream of CRL4CRBN. Consistent with this, both MLN4924 and MSO, which blocked GS ubiquitylation, rescued the effect of mTOR depletion by shRNA on GS stability (FIGS. 8F and 8G). Together, these results demonstrated that glutamine stimulates mTORC1-dependent phosphorylation of USP15, which is important for the binding of USP15 to GS to control GS stability.

### Example 8: To Identify the mTORC1 Phosphorylation Site on USP15

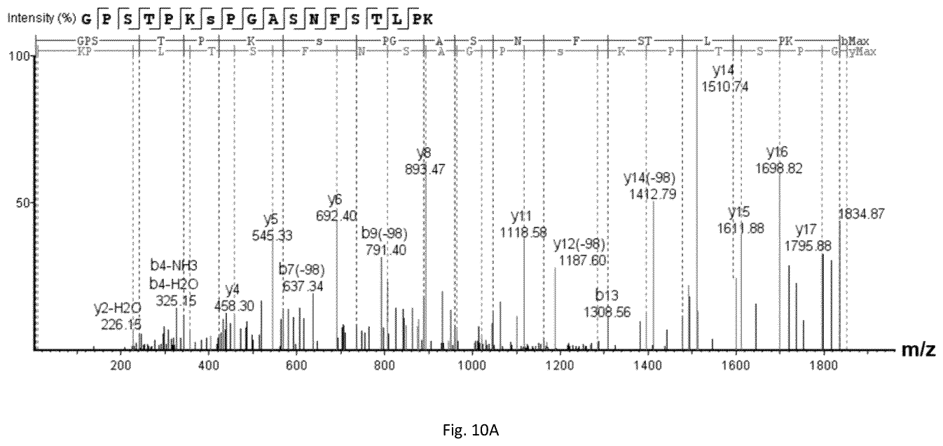

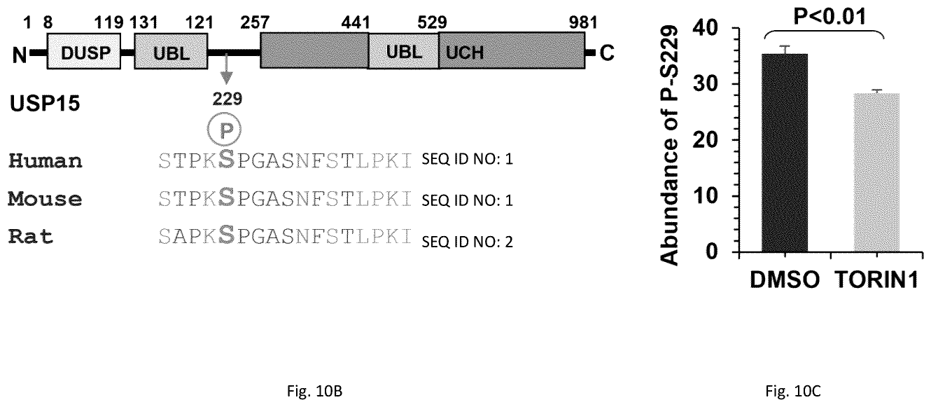

To map the phosphorylation sites of USP15, which has 32 potential serine/threonine phosphorylation sites reported in ‘PhosphoSitePlus’ with the most prevalent serine 229, USP15^{Flag} was immune-purified from 293FT cells treated with DMSO or 250 nM Torin1 for 16 h and analyzed by MS analysis. Known domains of USP15 are shown in FIG. 9. S229 was of particular interest, because the proteomic studies revealed that S229 was phosphorylated (FIG. 10A). Moreover, it represents an mTOR consensus sequence motif S/T-P (where S is serine; T is threonine; and P is proline) (FIG. 10B). S229 phosphorylation was significantly decreased in Torin1-treated cells (FIG. 10C). Thus, the MS data were consistent with the other results, showing that USP15 was phosphorylated at serine in an mTORC1-dependent manner (FIG. 7B-7C).

### Example 9: Summary

Based on the results of the other Examples, the CRL4^{CRBN}-p97 pathway is hereinafter called the CRL4^{CRBN}-USP15 pathway.

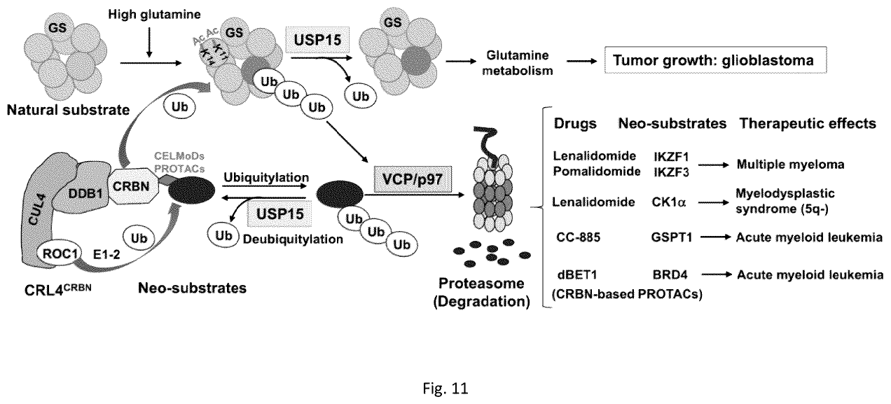

A proposed model for the role of USP15 in regulating the stability of natural substrate GS and neo-substrates in the CRL4^{CRBN}-USP15 pathway is illustrated in FIG. 11. High glutamine induces GS acetylation at lysines 11 and 14 to create a degron that binds CRBN. Subsequently, acetylated GS is ubiquitylated by CRL4^{CRBN} segregated by p97 and degraded by the proteasome. However, in glioblastoma (GBM) and other cancers, upregulation of USP15 stabilizes GS, a key metabolic enzyme in glutamine metabolism, which is essential for tumor growth. Most importantly, USP15 antagonizes CELMoD/PROTAC-induced ubiquitylation of target proteins, thereby inhibiting their subsequent degradation. This accounts for intrinsic resistance in IMiD-resistant MM cell lines that greatly overproduce USP15 protein. Inhibition of USP15 represents a valuable therapeutic target to potentiate CELMoD and CRBN-based PROTAC therapies for the treatment of cancer including MM, deletion 5q (del(5q)) subtype of myelodysplastic syndrome (MDS), acute myeloblastic leukemia (AML) and other human diseases.

In the present study, USP15 was identified as a GS-interacting partner based on two independent proteomic studies. The presented results provide the first direct evidence that USP15 deubiquitylates and stabilizes seven tested CRL4^{CRBN} protein targets, including GS, IKZF1, IKZF3, CK1α, RNF166, GSPT1 and BRD4 via natural substrate, molecular glue and PROTAC mechanisms. It functions downstream of CRL4^{CRBN} and upstream of p97 and proteasome. This accounts for intrinsic resistance of IMiDs in MM. These findings also indicate that USP15 protein expression is a potential biomarker of response or resistance to IMiD therapy in patients with MM.

USP15 was identified as a potential GS-interacting protein by two independent proteomic studies. Preliminary results demonstrated that endogenous USP15 interacts with endogenous GS, and deubiquitylates polyubiquitylated GS in vivo and in vitro (FIG. 6A-6L). Depletion of USP15 by the DUB inhibitors (FIG. 2A-2L), shRNA-mediated knockdown (FIG. 3A-3J), or CRISPR/Cas9 genome editing promotes GS ubiquitylation and subsequent degradation (FIGS. 4A-4M, 5A-5G, and 6A-6L). The sequential events of GS ubiquitylation and degradation were defined, and it was found that USP15 acts downstream of CRL4^{CRBN} and upstream of p97 and proteasome by counteracting the function of CRL4^{CRBN} to control GS stability.

### Example 10: Materials and Methods

### Materials and Cell Lines

Lenalidomide (Chem-Pacific), MLN4924 (Pevonedistat, from Active Biochem), MG132 and PR-619 (Sigma), Bortezomib (LC Laboratories), CB-5083 (Selleckchem), CC-885 (MedKoo Biosciences), Torin1, dBET1 and NSC632839 hydrochloride (Tocris) were dissolved in dimethyl sulfoxide (DMSO) at room temperature and were stored at −80° C. until use. L-Methionine sulfoximine (MSO) and cycloheximide (Sigma) were dissolved in distilled water and kept at −80° C. For DNA transfection, Fugene HD was from Promega.

HEK293 cells, 293FT cells, H1299 cells, MCF7 cells, MM.1S, U266, NCI-H929 and RPMI-8226 were purchased from ATCC (American Type Culture Collection, Manassas, VA, USA). KMS-11, ARP-1 and OPM-1 cell lines were provided by from a lab at the University of Pennsylvania, Philadelphia, PA, USA.

### Antibodies

Anti-glutamine synthetase (E-4; sc-74430), anti-IKZF1 (E-2, sc-398265), anti-IKZF3/Aiolos (3H5-G7; sc-293421) antibodies were from Santa Cruz Biotechnology. Mouse monoclonal anti-USP15 antibody (1C10, H00009958-M01) used for Western blot was from Abnova. Anti-ubiquitin (P4D1-All, 05-944), Anti-CRBN (HPA045910), anti-Flag HRP, and anti-Myc HRP antibodies were from Sigma. Anti-p97/VCP (ab11433), anti-GSPT1 (ab49878), anti-DDB1 (ab21080), anti-3-Actin HRP conjugated (AC-15, ab49900) antibodies were from Abcam. Anti-CUL4A (2699) antibody was from Cell Signaling Technology. Anti-BRD4 Antibody (BL-149-2H5; A700-004) was from Bethyl. Anti-Flag HRP conjugated antibody (600-403-383) was from Rockland Immunochemicals.

For secondary antibodies, HRP Goat Anti-Rabbit IgG (PI-1000) and HRP Horse Anti-Mouse IgG (PI-2000) were from Vector Laboratories.

For immunoprecipitation (IP), rabbit polyclonal anti-USP15 antibody (NB110-4069) was from Novus Biologicals. Rabbit IgG Isotype Control (normal rabbit IgG; sc-2027) was from Santa Cruz Biotechnology. EZview Red anti-Flag M2 (F2426) and EZview Red Anti-c-Myc Affinity Gels (E6654) were from Sigma.

### DNA Plasmids

Human USP15 expression vector pcDNA3.1+/C-(K)-DYK with a C-terminal Flag tag, expressing USP15 isoform 1 (NM_001252078.2) was purchased from GenScript. Human GS expression vector pCMV6-GS-Myc-Flag (C-terminal Myc-Flag tag) were purchased from OriGene. pCMV6-GS-Myc was generated by introducing a STOP codon between Myc and Flag by using a QuikChange Site-Directed Mutagenesis Kit (Stratagene, La Jolla, CA). The full-length cDNA of IKZF3 was amplified from the IKZF3 expression vector pcDNA3.2 with a N-terminal HA tag (a kind gift from Dr. Benjamin Ebert, Dana Farber Cancer Institute), and then subcloned into pCMV6-Flag-Myc tags (pCMV6-IKZF3Flag-Myc). All cDNAs cloned into mammalian expression vectors are confirmed by DNA sequencing.

Generation of Stable Cell Lines Expressing shRNAs Targeting USP15

For tetracycline- and doxycycline-inducible system, the TRIPZ inducible lentiviral shRNAs for human USP15 (clone ID #1: V2THS_5710; clone ID #2: V2THS_13437) were purchased from Horizon Discovery. The shRNAs were induced by using 2 μg/ml doxycycline for 3-4 days.

For GIPZ Lentiviral shRNA system, the GIPZ lentiviral constructs expressing nontargeting (control, CT) and human USP15 shRNAs (USP15_1 shRNA: V2LHS_196921; USP15_2 shRNA: V2LHS_5710; USP15_3 shRNA: V2LHS_13436; USP15_4 shRNA: V2LHS_13437; USP15_5 shRNA: V3LHS_336555; and USP15_6 shRNA: V3LHS_336551) were purchased from Horizon Discovery. Six lentiviruses in the GIPZ Lentiviral shRNA vectors targeting USP15 were screened to identify shRNAs that optimally suppressed USP15. Virus preparation and cell infection were performed according to the manufacturer's protocol, with minor modifications. Briefly, shRNA-encoding plasmids were co-transfected with psPAX2 (packaging plasmid) and pMD.2G (enveloping plasmid) into HEK293FT cells using Fugene HD (Promega). Virus-containing supernatants were harvested at 48 h and 72 h post transfection. The lentiviruses were precipitated using PEG-it virus precipitation solution according to the manufacturer's protocol (System Biosciences). Target cells were transduced with lentiviral particles in the presence of 8 μg/ml polybrene, followed by selection with puromycin (1 μg/ml) for multiple myeloma cell lines, and 2-4 μg/ml for 293 cells and 293FT cells) for 2 weeks. Knockdown efficiencies were analyzed by immunoblot.

### Generation of USP15 Knockout 293FT Cells by CRISPR Genome Editing

Cells, cultured in a 24-well plate, were transiently transfected with 0.5 μg of human USP15 CRISPR/Cas9 KO Plasmids (catalog #sc-402416; Santa Cruz Biotechnology) using Fugene HD (Promega). Two days after transfection, a single cell was seeded in 96-well plate via serial dilutions. After 2 weeks, single clones were obtained and expanded to validate the editing of USP15 by Western blot.

### Generation of Stable MM Cell Lines Expressing USP15^{Flag} and its Mutant

The protocol for generation of stable MM cell lines expressing USP15^{Flag} and its a catalytically inactive C298A-USP15^{Flag} mutant, constructed in pCDH-T2AcGFP-MSCV (System Biosciences), is based on recent work (Nguyen T V, et al., Mol Cell. 2016; 61(6):809-20). Human USP15 expression vector pcDNA3.1+USP15Flag (all tagged proteins are indicated by a superscripted tag either before or after the name to indicate tagging at the N- or C-terminus), USP15 isoform 1 (NM_001252078.2), was purchased from GenScript. Briefly, WT USP15^{Flag} and its mutant cDNAs will be amplified from pcDNA3.1+ USP15Flag and re-constructed in pCDH-T2AcGFP-MSCV.

### Immunoblot Analysis, Immunoprecipitation (IP) and TUBE2 Pulldown

The protocols for immunoblot analysis and IP were performed as described in T. V. Nguyen et al. (Mol Cell 61, 809-820 (2016); T. Van Nguyen et al., Molecular cell 45, 210-221 (2012)).

### IP USP15 in HEK 293FT Cells Cultured in 10-cm Plates

HEK293FT cells were cultured in 10-cm plates. Cells were starved of glutamine for 24 h and pretreated with the inhibitors before adding Q4 for 2 h. Cells were harvested by washing in cold PBS 2× and freeze down at −80° C. Cells were lysed with 1 ml BD150 (10 mM Tris [pH 7.5], 150 mM NaCl, 1% Triton X-100, and 1 mM DTT containing a protease inhibitor cocktail).

Antibodies used for the IP (immunoprecipitation) include rabbit polyclonal anti-USP15 Ab (1 μg/μl) using 4 μl and normal rabbit IgG control using 10 μl (0.4 μg/μl), which is an unconjugated, affinity purified isotype control immunoglobulin from rabbit in PBS with 0.1% sodium azide and 0.1% gelatin.

IP occurred for 2 hr. Then, 30 μl protein G beads were added for 1 h. IP buffer was used 3×, and elution occurred with 50 μl 1.5×SDS-SB.

The results were run on a 4-15% gel with anti-USP15m: M-IP1-6 3 μl 1×SDS M-Input 1-6 10 μl and anti-GSm: M-IP1-6 15 μl 1×SDS Input 1-6 (5 μl).

The results were re-run on a 4-15% gel with phospho antibodies M-IP1-6 15 μl-1×1×WCL 15 μl-1× and M-IP1-6 15 μl-M-1×WCL 15 μl-1×.

TABLE 1

| | | Anti-USP15 Ab (1 μg/μl) using 4 μl | | | | |
|---|---|---|---|---|---|---|
| | | | | PR-619 Dub | CB5083-p97 | MLN4924 Cul |
| | | | | inhibitor | inhibitor | inhibitor |
| IP Ab | rIgG | | | 20 μM | 10 μM | 2 μM |
| Q4 | − | − | + | + | + | + |
| Samples | 1 | 2 | 3 | 4 | 5 | 6 |

LN-229 cell lines were seeded in eight to ten 10-cm cell culture plates and cultured until they reached 80% confluence. Cells were washed with 1×PBS and lysed in 500 μl 1% NP40 lysis buffer (25 mM Tris pH 7.5, 150 mM NaCl, 1% NP40, 0.5 mM, phenylmethylsulfonylfluoride (PMSF), protease inhibitor cocktail (Roche)). Lysates were centrifuged at 13000 rpm for 10 minutes. Protein concentration was measured using Bradford assay (Bio-Rad Laboratories). For the first IP MS experiment 3 mg of protein was used and for the second 5 mg. The concentration of the USP15 antibody (NB110-40690, Novus Biologicals, UK) and of the normal Rabbit IgG control (12-370, Millipore, Germany) was 5 μg/mg of lysate. The mixture of antibody/lysate was left rotating gently at 4° C. overnight.

After 12 hours 100-120 μl of 50% A agarose beads (Thermoscientific, USA) were added to the samples and were incubated while rotating at 4° C. for four further hours. (Before use, the beads were washed with 1×PBS and centrifuged at 1000 rpm for 1 min).

The mixture (beads-antibody-protein sample) was washed 5 times with 0.1% Tween-20/PBS. (Washing: 500 μl 0.1% Tween-20 PBS, mix, centrifuge for 2 min at 1000 rpm/4° C.). The 1/10 of pulldown product: 301 of 2×SDS-loading buffers was added, boiled for 5 min at 95° C., 5 μl of the sample were loaded onto a reducing SDS-PAGE using standard methods, and the rest was loaded on another 10% gel in parallel for silver staining (Silver Staining Kit, SilverXpress, Invitrogen; according to the manufacturer's instructions)

### TUBE2 Pulldown

The protocol was described in T. V. Nguyen et al. (Proceedings of the National Academy of Sciences of the United States of America 114, 3565-3571 (2017)).

### In Vitro Deubiquitylation Assay

The assay was performed as described in T. V. Nguyen et al. (Proceedings of the National Academy of Sciences of the United States of America 114, 3565-3571 (2017)). Briefly, USP15^{-/-} 293FT cells will be treated with IMiDs or the PROTAC dBET1 in the presence of proteasome or p97 inhibitors to accumulate polyubiquitylated target proteins, purified by TUBE2 Pull-down as described in T. V. Nguyen et al. (Proceedings of the National Academy of Sciences of the United States of America 114, 3565-3571 (2017)). The beads containing polyubiquitylated proteins were mixed in 30 μl of ubiquitylation buffer, followed by addition of 1 μg recombinant USP15 (rUSP15) protein (catalog #E-594-050, R&D Systems) and incubated at 37° C. for 0.5 h. rUSP15-treated samples were mixed with 30 μl of 2×SDS sample buffer, boiled for 5 min and subjected to Western blot analysis.

For competitive ubiquitylation/deubiquitylation assay, IKZF3^{FM} was purified from USP15^{-/-} 293FT cells. An in vitro competitive ubiquitylation/deubiquitylation of IKZF3^{FM} was carried out for 1 h at 30° C. in the presence or absence of E1, E2, ^{HA}Ub and recombinant CLR4^{CRBN} complex purified from insect cells in a final volume of 30 in the presence of 50 μM lenalidomide and recombinant USP15 (rUSP15; 1 μg) were added. Reactions were stopped by mixing with equal amount of 2×SDS sample buffer and analyzed by SDS-PAGE and immunoblotting with antibodies against Flag, USP15 and CRBN.

### Cycloheximide Chase Experiments

Cells were seeded overnight in complete medium in 12-well plates, and then pre-treated with or without lenalidomide (10 μM) for 30 min, followed by addition of 100 μg/ml cycloheximide (CHX). At the indicated times following addition of CHX, samples were harvested for immunoblot analysis.

### Statistical Analysis

Data are presented as mean±one standard deviation (SD); p values were calculated using an unpaired two-tailed Student's t test in the Microsoft Excel software. P>0.05 was not significant whereas P<0.05 and P<0.01 means significant and very significant, respectively.

### In Vitro Pull-Down Assay

The assay was previously described (Sambrook J, Russell D W. CSH Protoc. 2006; 2006 (1). Epub 2006/01/01). Briefly, 1 μg of recombinant GST-tagged human GS protein (catalog #GLUL-4993H; Creative BioMart) and GST protein (control), immobilized on glutathione-Sepharose 4B beads (GE Healthcare), will be incubated with 1 μg recombinant His-tagged human USP15 Protein (catalog #USP15-358H, Creative BioMart) for 1 h at 4° C. in binding buffer: 20 mM Tris-HCl (pH 8.0), 200 mM KCl, 1 mM DTT, 5% glycerol, 1% Triton X-100 and proteinase inhibitors. After washing, bound proteins will be analyzed by IB.

### Peptide Pull-Down Assay

The assay will be performed as described in a recent study (Nguyen T V, et al., Mol Cell. 2016; 61(6):809-20). Biotinylated USP15 peptides (GS-binding region) and GS peptides (USP15-binding region) will be synthesized from Biomatik.

### Identification of USP15 Phosphorylation by Mass Spectrometry (MS)

MS analysis to identify the phosphorylation sites of USP15 was done at the Charles W. Gehrke proteomics center—University of Missouri, as described previously (Peterson T R, et al., Cell. 2009; 137(5):873-86. Epub 2009/05/19).

1. A method of treating cancer in a subject in need thereof, the method comprising: administering to the subject in need thereof a therapeutically effective amount of a USP15 inhibitor and at least one chemotherapeutic drug.

2. The method of claim 1, wherein the chemotherapeutic drug is an immunomodulatory drug (IMiD), cereblon E3 ligase modulator (CELMoD), or CRBN-based proteolysis-targeting chimaera (PROTAC).

3. The method of claim 1, wherein the chemotherapeutic drug is selected from the group consisting of thalidomide, lenalidomide, pomalidomide, CC-122, CC-220, and CC-885.

4. The method of claim 2, wherein the CRBN-based PROTAC is selected from the group consisting of ARV-825, dBETl, dBRD9, THAL-SNS-032, BJS-03-123, BSJ-02-162, BSJ-01-187, YKL-06-102, BETd-246, TL13-149, DD-04-015, MT-802, MS4078, GSK983, MD-224, and LI 81.

5. The method of claim 1, wherein the cancer is multiple myeloma, glioblastoma, deletion 5q subtype of myelodysplastic syndrome, or acute myeloblastic leukemia.

6. The method of claim 1, wherein the cancer is IMiD-resistant.

7. A method of diagnosing and treating resistance to IMiD therapy in a subject with multiple myeloma undergoing IMiD therapy, the method comprising:
a) measuring an amount of USP15 protein in the subject;
b) comparing the amount of USP15 protein in the subject to a reference;
c) diagnosing the subject with resistance to IMiD therapy if the amount of USP15 protein in the subject is higher than the reference; and
d) administering a USP15 inhibitor to the subject.

8. The method of claim 7, wherein measuring the amount of USP15 protein in the subject comprises using immunohistochemical (IHC) staining of USP15 protein in bone marrow sections of the subject.

9. The method of claim 7, wherein the reference is obtained from measuring an amount of USP15 protein from a different subject or subjects without multiple myeloma.

10. A method of regulating CRL4^{CRB}-USP15 pathway and glutamine synthetase levels in a subject comprising administering a USP15 inhibitor to the subject.

11. The method of claim 1, wherein the USP15 inhibitor is a nucleic acid or a chemical inhibitor.

12. The method of claim 11, wherein the nucleic acid is selected from the group consisting of an USP15 antisense mRNA, an USP15 siRNA, an USP15 shRNA, an USP15 miRNA, and an USP15 oligonucleotide.

13. The method of claim 11, wherein the chemical inhibitor is a deubiquitinating enzyme (DUB) inhibitor.

14. The method of claim 11, wherein the chemical inhibitor is selected from the group consisting of PR-619, NSC632839, and N-Ethylmaleimide (NEM).

15. The method of claim 11, wherein the chemical inhibitor is mitoxantrone or an ubiquitin variant.

16. The method of claim 1, wherein the subject is a mammal.

17. The method of claim 1, wherein the subject is a human, mouse, or rat.

18. The method of claim 7, wherein the USP15 inhibitor is a nucleic acid selected from the group consisting of an USP15 antisense mRNA, an USP15 siRNA, an USP15 shRNA, an USP15 miRNA, and an USP15 oligonucleotide or a chemical inhibitor selected from the group consisting of a deubiquitinating enzyme (DUB) inhibitor, PR-619, NSC632839, and N-Ethylmaleimide (NEM), mitoxantrone and an ubiquitin variant.

19. The method of claim 10, wherein the USP15 inhibitor is a nucleic acid selected from the group consisting of an USP15 antisense mRNA, an USP15 siRNA, an USP15 shRNA, an USP15 miRNA, and an USP15 oligonucleotide or a chemical inhibitor selected from the group consisting of a deubiquitinating enzyme (DUB) inhibitor, PR-619, NSC632839, and N-Ethylmaleimide (NEM), mitoxantrone and an ubiquitin variant.


SEQUENCE LISTING (2 sequences)

SEQ ID NO: 1 - protein, 17 aa, Homo sapiens
STPKSPGASNFSTLPKI

SEQ ID NO: 2 - protein, 17 aa, Rattus norvegicus
SAPKSPGASNFSTLPKI